(19) United States
(12) Patent Application Publication
(10) Pub. No.: US 2022/0142169 A1
(43) Pub. Date: May 12, 2022

(54) INHIBITING INSECTICIDE RESISTANCE AND MAKING SUSCEPTIBLE INSECTS HYPER-SUSCEPTIBLE TO PESTICIDES

(71) Applicant: Board of Trustees of Michigan State University, East Lansing, MI (US)

(72) Inventor: Barry Robert Pittendrigh, Okemos, MI (US)

(21) Appl. No.: 17/428,255

(22) PCT Filed: Feb. 7, 2020
(86) PCT No.: PCT/US2020/017351

§ 371 Date: Aug. 3, 2021

Related U.S. Application Data
(60) Provisional application No. 62/802,305, filed on Feb. 7, 2019.

(51) Int. Cl. A01N 59/08; A01P 7/04; A01N 59/02; C12N 15/82

(52) U.S. Cl. CPC A01N 59/08; A01P 7/04; C12N 15/8286; C12N 15/8218; A01N 59/02

(57) ABSTRACT

Insecticide resistance is an ongoing challenge in agriculture and vector control. Here, we demonstrate a novel strategy to attenuate resistance. One embodiment provides a method for increasing susceptibility of an insect to a pesticide comprising contacting an insect, soil, wood, plant, seeds, grain or manmade structure with one or more inhibitors of insect resistance.

### PRIORITY

This application claims the benefit of priority to U.S. Provisional Patent Application No. 62/802,305, filed on Feb. 7, 2019, the disclosure of which is incorporated herein by reference in its entirety.

### BACKGROUND OF THE INVENTION

Pesticide insecticide resistance is an ongoing challenge for agricultural production and vector borne disease control.

### SUMMARY OF THE INVENTION

Provided herein are methods of using inhibitors to protect against or treat pest infestation, and compositions and kits.

One embodiment provides a method for increasing susceptibility of an insect to a pesticide comprising contacting an insect, soil, wood, plant, seeds, grain or manmade structure with one or more inhibitors of insect resistance.

Another embodiment provides a method of decreasing resistance of an insect to a pesticide comprising contacting said insect, soil, wood, plant, seeds, grain or manmade structure with one or more inhibitors of insect resistance.

A further embodiment provides a method to increase toxicity of a pesticide in an insect comprising contacting said insect, soil, wood, plant, seeds, grain or manmade structure with one or more inhibitors of insect resistance.

Another embodiment provides a method for providing protection against or treating a pest infestation comprising contacting said pest, soil, wood, plant, seeds, grain or manmade structure with one or more inhibitors of insect resistance.

One embodiment provides a method for reducing insect resistance in a plant comprising expressing in said plant an RNA that specifically interferes with expression of an insect gene.

In one embodiment, the inhibitor contacts said insect, soil, wood, plant, seeds, grain or manmade structure before, after or simultaneously with one or more pesticides.

In another embodiment, the inhibitor modulates the activity of proteins that play a role in energy pathways or other metabolic pathways of said insect, including energy-related pathway proteins, metabolism-related pathway proteins, insulin/insulin-like growth factor (IGF)-like signaling (US) pathway proteins; insulin signaling pathway proteins, including Phosphoenolpyruvate carboxykinase (PEPCK), Glycogen synthase kinase 3 beta (GSK3β), Lipin (Lpin-PE), Insulin-like peptide 6 (Dilp6-PD), Cchamide-2 (CCHa2-PA), Insulin-like peptide 8 (Dilp8-PB), Flotillin (Flo2-PJ), rolled (rl-PH), Phosphorylase kinase gamma subunit (PhKγ-PF), Hexokinase (Hex-C-PA), Fructose-1,6-bisphosphatase (fbp-PF), Lipin (Lpin-PL), Acetyl-coa carboxylase/biotin carboxylase 1 (ACC-PA), Glycogen synthase (GlyS-PA), and/or Glycogen phosphorylase (GlyP-PA).

In one embodiment, the inhibitor is one or more of hydrazine sulphate, 3-alkyl-1,8-dibenzylxanthines, oxalate and phosphonoformate, 3-mercaptopicolinic acid, (N′1-({5-[1-methyl-5-(trifluoromethyl)-1H-pyrazol-3-yl]-2-thienyl}methylidene)-2,4-dichlorobenzene-1-carbohydrazide), metformin, Beryllium, copper, lithium chloride, dibromocantharelline, hymenialdesine, meridianin, sodium borate, and/or resorcylic acid lactone. In another embodiment, the inhibitor is hydrazine sulphate and/or lithium chloride.

In one embodiment, the insect or pest is cotton bollworm, tobacco whitefly, two-spotted spider mite, diamondback moth, taro caterpillar, red flour beetle, green peach aphid, fall armyworm, bedbugs, cockroaches, ants, termites, mites, head or body lice, rice weevils, maize weevils, fly, and/or cotton aphid. In another embodiment, the insect or pest is fall armyworm, spotted wing Drosophila, red flour beetles, and/or diamondback moths.

In one embodiment, the plant is a dicotyledon or monocotyledon, including a crop, flower, or forestry plant.

In one embodiment, the gene codes for a protein that has a role in an energy pathway or metabolic pathway of said insect, including energy-related pathway proteins, metabolism-related pathway proteins, insulin/insulin-like growth factor (IGF)-like signaling (IIS) pathway proteins; insulin signaling pathway proteins, including Phosphoenolpyruvate carboxykinase (PEPCK), Glycogen synthase kinase 3 beta (GSK3p), Lipin (Lpin-PE), Insulin-like peptide 6 (Dilp6-PD), Cchamide-2 (CCHa2-PA), Insulin-like peptide 8 (Dilp8-PB), Flotillin (Flo2-PJ), rolled (rl-PH), Phosphorylase kinase gamma subunit (PhKT-PF), Hexokinase (Hex-C-PA), Fructose-1,6-bisphosphatase (fbp-PF), Lipin (Lpin-PL), Acetyl-coa carboxylase/biotin carboxylase 1 (ACC-PA), Glycogen synthase (GlyS-PA), and/or Glycogen phosphorylase (GlyP-PA). In one embodiment, the gene is PEPCK-PA (Accession numbers: FLYBASE:FBgn0003067; BT003447.1 (mRNA); AA039450.1 (protein); AE013599.5 (gene)) or GSK3β-PO (Accession numbers: Chromosome 3R, NT_033777.3 (30022842 . . . 30035311); Chromosome 3R, NT_033777.3 (30022842 . . . 30035311)).

In one embodiment, the contacting is by spraying or in a bait as a liquid or powder on said insect, soil, wood, plant, seeds, grain or manmade structure or by ingestion by the insect and/or pest.

One embodiment provides a composition comprising at least one inhibitor of insect resistance, at least one pesticide and a carrier.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1. Reverse transcription quantitative PCR (RT-qPCR) validation of fifteen transcripts in insulin signaling pathways putatively differentially expressed between DDT-resistant 91-R and the -susceptible 91-C strain [FDR<0.05 and log 2(fold change) ≥|1.0|]. The y-axis on the left and right indicate the relative gene expression level of 91-R versus 91-C based on RT-qPCR-based and RNA-seq-based estimates, respectively. Full name of genes: rl-PH, rolled; PhKγ-PF, Phosphorylase kinase gamma subunit; Hex-C-PA, Hexokinase; fbp-PF, Fructose-1,6-bisphosphatase I; Lpin-PL, Lipin; ACC-PA, Acetyl-coa carboxylase/biotin carboxylase 1; GlyS-PA, Glycogen synthase; GlyP-PA, Glycogen phosphorylase; Lpin-PE, Lipin; Dilp6-PD, Insulin-like peptide 6; CCHa2-PA, Cchamide-2; Dilp8-PB, Insulin-like peptide 8; GSK3β-PO, Glycogen synthase kinase 3 beta; PEPCK-PA, Phosphoenolpyruvate carboxykinase; Flo2-PJ, Flotillin.

FIG. 2. Alignment of deduced amino acid sequences for Phosphoenolpyruvate carboxykinase 2_PA from D. melanogaster strains Canton-S, 91-C, and 91-R.

FIG. 3. Alignment of deduced amino acid sequences for glycogen synthase kinase 3 beta_PM from D. melanogaster strains Canton-S, 91-C, and 91-R.

FIGS. 4A-B. Lifespan of 91-C and 91-R. A: longevity of females of 91-C and 91-R. Median lifespan for 91-C♀ and 91-R♀ is 72.18 (95% CI, 67.36-77.34) and 85.29 (95% CI, 80.45-90.79) days, respectively; B: longevity of males of 91-C and 91-R. Median lifespan for 91-C♂ and 91-R♂ is 70.58 (95% CI, 64.45-77.12) and 91.80 (95% Cl, 85.81-99.39) days, respectively.

FIGS. 5A-F. Survival of 91-C and 91-R females and males after starvation. (A) Survival of 3-4 days old females of 91-C and 91-R. The median survival is 122.26 (95% CI: 119.52-125.76) and 106.37 (95% CI: 104.72-108.17) hours, respectively. (B) Survival of 5-6 days old females of 91-C and 91-R. The median survival is 120.33 (95% CI: 117.51-123.79) and 116.33 (95% CI: 114.00-119.08) hours, respectively. (C) Survival of 9-10 days old females of 91-C and 91-R. The median survival is 102.82 (95% CI: 100.70-105.15) and 81.99 (95% CI: 80.34-83.66) hours, respectively. (D) Survival of 3-4 days old males of 91-C and 91-R. The median survival is 83.85 (95% CI: 82.84-84.86) and 70.13 (95% CI: 69.23-71.03) hours, respectively. (E) Survival of 5-6 days old males of 91-C and 91-R. The median survival is 74.83 (95% CI: 73.44-76.20) and 64.86 (95% CI: 63.96-65.76) hours, respectively. (F) Survival of 9-10 days old males of 91-C and 91-R. The median survival is 86.93 (95% CI: 85.70-88.19) and 78.94 (95% CI: 77.99-79.90) hours, respectively.

FIG. 6. Glycogen content of 91-C and 91-R flies per mg of fly (fresh weight) after starvation. Data are shown as means t SEM. *, P<0.05; NS, no significant difference.

FIG. 7. Differentially expressed genes shown in insulin signaling pathway. Full name of genes: up-regulated: ACC, Acetyl-CoA carboxylase; GK, hexokinase C; FBP, Fructose-1,6-bisphosphatase; GYS, glycogen synthase; PHK, Phosphorylase kinase; PYG, glycogen phosphorylase; ERK1/2, rolled (rl); Lipin; down-regulated: Flotillin; INS, Insulin-like peptide; PEPCK, Phosphoenolpyruvate carboxykinase; GSK3β, Glycogen synthase kinase 3 beta; Lipin.

FIGS. 8A-F. Cypermethrin without (control) and with inhibitor feeding of Hydrazine sulfate (Hys) or Lithium Chloride (LiCl).

### DETAILED DESCRIPTION OF THE INVENTION

Disclosed herein is the discovery that insect genes can be targeted to improve susceptibility to pesticides. For example, pesticide resistance is an ongoing problem in the control of insects that are agricultural pests or vector of diseases. Several insecticide classes, for example, pyrethroids that share a mode of action with DDT, are being lost as control agents due to resistance; pyrethroids are the mainstay of indoor residual spraying and insecticide-treated bed nets. There is a lack of new insecticides with novel modes of action. Here we present a proof-of-principle experiment providing a path forward in identifying Achilles's heel mechanisms allowing the re-use and extended longevity of irreplaceable pest control agents, as evidenced by an inhibitor of an insulin/insulin-like growth factor (IGF)-like signaling (IIS) pathway gene, which virtually eliminated resistance in a highly DDT-resistant strain.

### Definitions

For the purposes of clarity and a concise description, features can be described herein as part of the same or separate embodiments; however, it will be appreciated that the scope of the invention may include embodiments having combinations of all or some of the features described.

The terminology used herein is for the purpose of describing particular embodiments only and is not intended to be limiting of the invention.

As used herein, the indefinite articles “a”, “an” and “the” should be understood to include plural reference unless the context clearly indicates otherwise.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the term “about” means plus or minus 10% of the indicated value. For example, about 100 means from 90 to 110.

The compounds and compositions provided herein can “protect against or treat pest infestation.” The term refers to affecting a pest's ability to infest and, therefore, refers to the inhibition or elimination of pest infestation. The term is also meant to include a reduction in the damage caused by the pest and/or the ability of the pest to infest and/or cause damage. The terms “infest” or “infestation” are generally used interchangeably throughout. Therefore, in the methods as described herein, the pest's ability to infest or maintain an infestation is inhibited or eliminated. “Effective amounts” for achieving any of the desired endpoints described herein, such as protecting against or treating pest infestation refers to any amount that results in any of the above. A skilled person is able to determine such amounts with methods known in the art.

As used herein, the term “plant” is not particularly limited, as long as the “plant” can be infested by insects (e.g., Lepidoptera), such as various crops, flower plants, or forestry plants. The plant may be (but is not limited to): dicotyledon, monocotyledon or gymnosperms. More specifically, the plants may include (but are not limited to): cotton, wheat, barley, rye, rice, corn, sorghum, sugar beet, apple, pear, plum, peach, apricot, cherry, strawberry, raspberry, blackberry, beans, lentils, peas, soybeans, rapeseed, mustard, poppy, oleanolic, sunflowers, coconut, castor oil plants, cocoa beans, peanuts, gourd, cucumber, watermelon, flax, hemp, jute, oranges, lemons, grapes grapefruit, spinach, velvetleaf lettuce, asparagus, cabbage, Chinese cabbage, Chinese cabbage, carrots, onions, potatoes, tomatoes, green peppers, avocados, cinnamon, camphor, tobacco, nuts, coffee, eggplant, sugar cane, tea, pepper, vines Oyster Asakusa, bananas, natural rubber trees and ornamental plants, etc.

As used herein, the terms “including”, “includes”, “having”, “has”, “with”, or variants thereof, are intended to be inclusive similar to the term “comprising.”

As used herein, said “contain”, “have” or “including” include “comprising”, “mainly consist of”, “basically consist of” and “formed of”; “primarily consist of”, “generally consist of” and “comprising of” belong to generic concept of “have” “include” or “contain”.

### Inhibitors of Resistance/Increase Susceptibility

As proof of concept, using genomic tools to compare DDT susceptible and resistant Drosophila melanogaster (hereafter referred to as Drosophila) populations, it was identified that Phosphoenolpyruvate carboxykinase (PEPCK) and Glycogen synthase kinase 3 beta (GSK3β), when inhibited respectively with hydrazine sulphate or lithium chloride, both caused a dramatic reduction in resistance in DDT resistant Drosophila. Such inhibition (e.g., with hydrazine sulphate) can result in a >200-times reduction in resistance to DDT. Additionally, these aforementioned example inhibitors can also increase the toxicity of DDT in susceptible insects (upwards of 10-times increased toxicity of the pesticide). This model system demonstrates that PEPCK or GSK30 can be targeted to inhibit insects' response to pesticides. These are practical demonstrations of the invention that one can inhibit proteins in pathways that play a role in the ability of insects to be tolerant of or resistant to pesticides. Such proteins are called Achilles' heel resistance traits. As an example, Table 4 demonstrates that exposing DDT resistant and susceptible Drosophila to the aforementioned inhibitors alters the LD₅₀ in each of these fly strains. These studies suggest the potential for the use of such inhibitors (e.g., hydrazine sulphate or lithium chloride) to be incorporated with insect baits that also contain pesticides or PEPCK or GSK3β represent relevant target sites for compounds that can be sprayed with, before or after insecticide exposure.

The inhibitors make pesticide/insecticide-resistant pests/insects (those insects that are less susceptible than others to the pesticide/insecticide) susceptible to the pesticide/insecticide being applied before, after or during the application of the inhibitor and the inhibitors also make pesticide/insecticide susceptible insects hyper-susceptible to the pesticide/insecticide (increase toxicity of the pesticide/insecticide).

Chemical Agents

Chemical inhibitors which decrease resistance/increase susceptibility/toxicity of a pesticide/insecticide can include any agent that modulates/interferes with the activity, including inhibits the activity, of proteins that play a role in energy pathways or other metabolic pathways of an organism, including, but not limited to, energy-related pathway components, metabolism-related proteins, insulin/insulin-like growth factor(IGF)-like signaling (IIS) pathway proteins; insulin signaling pathway proteins, including such proteins like Phosphoenolpyruvate carboxykinase (PEPCK), Glycogen synthase kinase 3 beta (GSK3β), Lipin (Lpin-PE), Insulin-like peptide 6 (Dilp6-PD), Cchamide-2 (CCHa2-PA), Insulin-like peptide 8 (Dilp8-PB), Flotillin (Flo2-PJ), rolled (rl-PH), Phosphorylase kinase gamma subunit (PhKγ-PF), Hexokinase (Hex-C.-PA), Fructose-1,6-bisphosphatase (fbp-PF), Lipin (Lpin-PL), Acetyl-coa carboxylase/biotin carboxylase 1 (ACC-PA), Glycogen synthase (GlyS-PA), and/or Glycogen phosphorylase (GpyP-PA) activity, which would lead to increase susceptibility to pesticides/insecticides and/or reduced resistance to pesticides/insecticides.

Inhibitors for use in the Invention can include those provided below:

| | Gene | CDS | |
|---|---|---|---|
| Gene | location | length | inhibitors |
| Under expressed genes | | | |
| PEPCK-PA | 2R:18,527,430- | 1917 | Hydrazine sulphate |
| | 18,535,749 | | 3-alkyl-1,8- |
| | | | dibenzylxanthines |
| | | | oxalate and |
| | | | phosphonoformate |
| | | | 3-mercaptopicolinic |
| | | | acid |
| | | | (N'1-({5-[1-methyl- |
| | | | 5-(trifluoromethyl)- |
| | | | 1H-pyrazol-3-yl]-2- |
| | | | thienyl}methylidene)- |
| | | | 2,4- |
| | | | dichlorobenzene-1- |
| | | | carbohydrazide) |
| | | | metformin |
| GSK3β-PO | X:2,633,952- | 1326 | Beryllium |
| | 2,679,553 | | |
| | | | Lithium Chloride |
| | | | (IC50 = 2mM) |
| | | | Dibromocantharelline |
| | | | (IC50 = 3 μM) |
| | | | Hymenialdesine |
| | | | (IC50 = 10 nM) |
| | | | Indirubin (IC50 = 5- |
| | | | 50 nM) |
| | | | Meridianin |
| Lpin-PE | 2R:8,136,986- | 3138 | sodium borate |
| | 8,156,654 | | |
| Over expressed genes | | | |
| Lpin-PL | 2R:8,136,986- | 2979 | sodium borate |
| | 8,156,654 | | |
| Hex-C | 2R:15,218,837- | 1365 | |
| | 15,220,329 | | |
| ERK | 2R:1,071,462- | 800 | FR180204; |
| (rl)-PH | 1,125,927 | | resorcylic acid |
| | | | lactone |
| | | | (RAL) FR148083 |

RNAi

Transgenic plants expressing RNAi insect-specific constructs to knockdown Achilles' heel resistance traits in insects can also be used, in for example, a two-part control system. Thus, as insects consume the host plant, RNAi impacts the Achilles' heel resistance trait(s) rendering the insect populations less resistant or more susceptible to pesticide sprays or other transgenic biopesticides. Thus, some embodiments of the invention provide the use of RNAi to knock down insect genes, thereby reducing resistance in the insect population, or making the insects much more susceptible to another toxin.

Herein the term “RNA interference” (RNAi) refers to blocking, using certain double-stranded RNAs, the expression of specific genes in vivo, facilitating mRNA degradation, and inducing cells to exhibit specific gene deletion phenotype. This process is also referred to as RNA intervention or interference.

In the present invention, the basic principles of RNA interference are as follows: using plants as an intermediate, insects would ingest plants expressing interfering RNAs capable of interfering with insect genes (such as RNAi designed to interfere genes in energy pathways or other metabolic pathways of an organism, including but not limited to, energy-related pathway component genes, energy metabolism-related genes, insulin/insulin-like growth factor (IGF)-like signaling (IIS) pathway genes; insulin signaling pathway genes, including such genes like Phosphoenolpyruvate carboxykinase (PEPCK); Glycogen synthase kinase 3 beta (GSK3β), Lipin (Lpin-PE), Insulin-like peptide 6 (Dilp6-PD), Cchamide-2 (CCHa2-PA), Insulin-like peptide 8 (Dilp8-PB), Flotillin (Flo2-PJ), rolled (rl-PH), Phosphorylase kinase gamma subunit (PhKγ-PF), Hexokinase (Hex-C-PA), Fructose-1,6-bisphosphatase (fbp-PF), Lipin (Lpin-PL), Acetyl-coa carboxylase/biotin carboxylase 1 (ACC-PA), Glycogen synthase (GlyS-PA), and/or Glycogen phosphorylase (GlyP-PA) expression, which would lead to increase susceptibility to pesticides/insecticides and/or reduced resistance to pesticides/insecticides. In particular, using gene transfection methods to express double-stranded RNAs (dsRNAs) of insect genes (full- or partial-length) in plant to produce interfering RNAs in plants (methods for producing transgenic plants can be found in Transgenic Plants: Methods and Protocols (Methods in Molecular Biology), Humana Press, 2004 or are otherwise known to those of ordinary skill in the art). When insects ingest such transgenic plants, interfering RNAs are ingested simultaneously. After entering inside insect bodies, the interfering RNAs can in turn inhibit the expression of insect genes.

Herein, the term “interfering molecules” generally refers to a kind of substance having insect prevention activity obtained from preparing or processing (such as in vivo processing) insect genes or their fragments (truncated form) as targets based on the present invention. Said “interfering molecules” include, for example, dsRNA, antisense nucleic acid (nucleotide), small interfering RNA, miRNA, etc.

As used herein, the term “dsRNA” refers to a double-stranded RNA molecule, which can degrade specific mRNA by targeting mRNA with homologous complementary sequences. This process is referred to as RNA interference pathway.

As used herein, “sufficiently complementary” refers to nucleotide sequences being sufficiently complementary, which can interact with each other in a predictable manner, such as forming secondary structure (such as stem-loop structure). Usually, there is at least 70% of nucleotides are complementary between two “sufficiently complementary” nucleotide sequences including, at least 80% of nucleotides are complementary; such as, at least 90% of nucleotides are complementary; including, at least 95% of nucleotides are complementary; for example, 98%, 99% or 100%.

Methods of determining sequence identity are conventional for skilled persons in the art, including using Blast software and EMBOSS software (The European Molecular Biology Open Software Suite (2000), Rice, P. Longden, I. and Bleasby, A. Trends in Genetics 16, (6) pp 276-277). As used herein, the term “identity” refers to the relationship between sequences at the nucleic acid or amino acid level. Through comparing the sequences (such as, two or more) with optimal alignment in the comparison window, “identity percentage” can be determined. Herein, the comparison between said sequences in the comparison window and the reference sequence with optimal sequence alignment may contain insertion or deletion. Said reference sequences do not contain insertion or deletion. Said reference window is selected from at least 10 consecutive nucleotides up to about 50, about 100, or up to about 150 nucleotides, including about 50-150 nucleotides. Then, by detecting the number of identical nucleotides between sequences in said window and divided the above number by the number of nucleotides in said window and multiplied by 100 to calculate “identity percentage.”

### Insects/Pests

The invention directed towards making insects and other pests, such as worms, more susceptible to pesticides/insecticides, such as make resistant insects susceptible (e.g. reduce resistance) to the insecticide and susceptible insects more so (e.g., super/hyper susceptible; more toxic to all insects).

In some embodiments of the invention the insect may include one or more of the following pests: cotton bollworm, tobacco whitefly, two-spotted spider mite, diamondback moth, taro caterpillar, red flour beetle, green peach aphid, fall armyworm, flies, and/or cotton aphid (stateoftheworldsplants.org/2017/report/SOTWP_2017_10_plant_health_state_of_research.pdf; see pages 66-67); in particular, one or more of fall armyworm, spotted wing Drosophila, red flour beetles, and/or diamondback moths.

In some embodiments of the invention the insect may be one or more of the following:

(1) an insect which is a plant pest, such as, but not limited, to Nilaparvata spp. (e.g. N. lugens (brown planthopper)); Laodelphax spp. (e.g. L. striatellus (small brown planthopper)); Nephotettix spp. (e.g. N. virescens or N. cincticeps (green leafhopper), or N. nigropictus (rice leafhopper)); Sogatella spp. (e.g. S. furcifera (white-backed planthopper)); Blissus spp. (e.g. B. leucopterus leucopterus (chinch bug)); Scotinophora spp. (e.g. S. vermidulate (rice blackbug)); Acrosternum spp. (e.g. A. hilare (green stink bug)); Parnara spp. (e.g. P. guttata (rice skipper)); Chilo spp. (e.g. C. suppressalis (rice striped stem borer), C. auricilius (gold-fringed stem borer), or C. polychrysus (dark-headed stem borer)); Chilotraea spp. (e.g. C. polychrysa (rice stalk borer)); Sesamia spp. (e.g. S. inferens (pink rice borer)); Tryporyza spp. (e.g. T. innotata (white rice borer), or T. incertulas (yellow rice borer)); Cnaphalocrocis spp. (e.g. C. medinalis (rice leafroller)); Agromyza spp. (e.g. A. oryzae (leafminer), or A. parvicornis (corn blot leafminer)); Diatraea spp. (e.g. D. saccharalis (sugarcane borer), or D. grandiosella (southwestern corn borer)), Narnaga spp. (e.g. N. aenescens (green rice caterpillar)); Xanthodes spp. (e.g. X. transversa (green caterpillar)); Spodoptera spp. (e.g. S. frugiperda (fall armyworm), S. exigua (beet armyworm), S. litura (Oriental leafworm), S. littoralis (climbing cutworm) or S. praefica (western yellowstriped armyworm)); Mythimna spp. (e.g. Mythmna (Pseudaletia) seperata (armyworm)); Helicoverpa spp. (e.g. H. zea (corn earworm), H. armigera); Colaspis spp. (e.g. C. brunnea (grape colaspis)); Lissorhoptrus spp. (e.g. L. oryzophilus (rice water weevil)); Echinocnemus spp. (e.g. E. squamos (rice plant weevil)); Diclodispa spp. (e.g. D. armigera (rice hispa)); Oulema spp. (e.g. O. oryzae (leaf beetle); Sitophilus spp. (e.g. S. oryzae (rice weevil)); Pachydiplosis spp. (e.g. P. oryzae (rice gall midge)); Hydrellia spp. (e.g. H. griseola (small rice leafminer), or H. sasakii (rice stem maggot)); Chlorops spp. (e.g. C. oryzae (stem maggot)); Diabrotica spp. (e.g. D. virgifera (western corn rootworm), D. barberi (northern corn rootworm), D. undecimpunctata howardi (southern corn rootworm), D. virgifera zeae (Mexican corn rootworm); D. balteata (banded cucumber beetle)); Ostrinia spp. (e.g. O. nubilalis (European corn borer)); Agrotis spp. (e.g. A. ipsilon (black cutworm)); Elasmopalpus spp. (e.g. E. lignosellus (lesser cornstalk borer)); Melanotus spp. (wireworms); Cyclocephala spp. (e.g. C. borealis (northern masked chafer), or C. immaculata (southern masked chafer)); Phaedon spp. (e.g. P. cochleariae (mustard leaf beetle)); Epilachna spp. (e.g. E. varivestis (Mexican bean beetle)); Popillia spp. (e.g. P. japonica (Japanese beetle)); Chaetocnema spp. (e.g. C. pulicaria (corn flea beetle)); Sphenophorus spp. (e.g. S. maidis (maize billbug)); Rhopalosiphum spp. (e.g. R. maidis (corn leaf aphid)); Anuraphis spp. (e.g. A. maidiradicis (corn root aphid)); Melanoplus spp. (e.g. M. femurrubrum (redlegged grasshopper) M. differentialis (differential grasshopper) or M. sanguinipes (migratory grasshopper)); Hylemya spp. (e.g. H. platura (seedcorn maggot)); Anaphothrips spp. (e.g. A. obscrurus (grass thrips)); Solenopsis spp. (e.g. S. milesta (thief ant)); or spp. (e.g. T. urticae (twospotted spider mite), T. cinnabarinus (carmine spider mite); Helicoverpa spp. (e.g. H. zea (corn earworm), or H. armigera (cotton bollworm)); Pectinophora spp. (e.g. P. gossypiella (pink bollworm)); Earias spp. (e.g. E. vittella (spotted bollworm)); Heliothis spp. (e.g. H. virescens (tobacco budworm)); Anthonomus spp. (e.g. A. grandis (boll weevil)); Pseudatomoscelis spp. (e.g. P. seriatus (cotton fleahopper)); Trialeurodes spp. (e.g. T. abutiloneus (banded-winged whitefly) T. vaporariorum (greenhouse whitefly)); Bemisia spp. (e.g. B. argentifolii (silverleaf whitefly)); Aphis spp. (e.g. A. gossypii (cotton aphid)); Lygus spp. (e.g. L. lineolaris (tarnished plant bug) or L. hesperus (western tarnished plant bug)); Euschistus spp. (e.g. E. conspersus (consperse stink bug)); Chlorochroa spp. (e.g. C. sayi (Say stinkbug)); Nezara spp. (e.g. N. viridula (southern green stinkbug)); Thrips spp. (e.g. T. tabaci (onion thrips)); Frankliniella spp. (e.g. F. fusca (tobacco thrips), or F. occidentalis (western flower thrips)); Leptinotarsa spp. (e.g. L. decemlineata (Colorado potato beetle), L. junta (false potato beetle), or L. texana (Texan false potato beetle)); Lema spp. (e.g. L. trilineata (three-lined potato beetle)); Epitrix spp. (e.g. E. cucumeris (potato flea beetle), E. hirtipennis (flea beetle), or E. tuberis (tuber flea beetle)); Epicauta spp. (e.g. E. vittata (striped blister beetle)); Empoasca spp. (e.g. E. fabae (potato leafhopper)); Myzus spp. (e.g. M. persicae (green peach aphid)); Paratrioza spp. (e.g. P. cockerelli (psyllid)); Conoderus spp. (e.g. C. falli (southern potato wireworm), or C. vespertinus (tobacco wireworm)); Phthorimaea spp. (e.g. P. operculella (potato tuberworm)); Macrosiphum spp. (e.g. M. euphorbiae (potato aphid)); Thyanta spp. (e.g. T. pallidovirens (redshouldered stinkbug)); Phthorimaea spp. (e.g. P. operculella (potato tuberworm)); Keiferia spp. (e.g. K. lycopersicella (tomato pinworm)); Limonius spp. (wireworms); Manduca spp. (e.g. M. sexta (tobacco hornworm), or M. quinquemaculata (tomato hornworm)); Liriomyza spp. (e.g. L. sativae, L. trifolli or L. huidobrensis (leafminer)); Drosophila spp. (e.g. D. simulans, D. yakuba, D. pseudoobscura, D. virilis or D. melanogaster (fruitflies)); Atherigona spp. (e.g. A. soccata (shoot fly); Carabus spp. (e.g. C. granulatus); Chironomus spp. (e.g. C. tentanus); Ctenocephalides spp. (e.g. C. felis (cat flea)); Diaprepes spp. (e.g. D. abbreviatus (root weevil)); Ips spp. (e.g. I. pini (pine engraver)); Tribolium spp. (e.g. T. castaneum (red floor beetle)); Glossina spp. (e.g. G. morsitans (tsetse fly)); Anopheles spp. (e.g. A. gambiae str. PEST (malaria mosquito) or A. albimanus (malaria mosquito); Acyrthosiphon spp. (e.g. A. pisum (pea aphid)); Apis spp. (e.g. A. melifera (honey bee)); Homalodisca spp. (e.g. H. coagulata (glassy-winged sharpshooter)); Aedes spp. (e.g. Ae. aegypti (yellow fever mosquito)); Bombyx spp. (e.g. B. mori (silkworm)); Locusta spp. (e.g. L. migratoria (migratory locust)); Boophilus spp. (e.g. B. microplus (cattle tick))s Acanthoscurria spp. (e.g. A. gomesiana (red-haired chololate bird eater)); Diploptera spp. (e.g. D. punctata (pacific beetle cockroach)); Heliconius spp. (e.g. H. erato (red passion flower butterfly), H. melpomene (postman butterfly) or H. himera); Plutella spp. (e.g. P. xylostella (diamontback moth)); Armigeres spp. (e.g. A. subalbatus); Culicoides spp. (e.g. C. sonorensis (biting midge)); Biphyllus spp. (e.g. B. lunatus (skin beetle)); Mycetophagus spp (e.g. M. quadripustulatus); Hydropsyche spp (caddisflies); Oncometopia spp. (e.g. O. nigricans (sharpshooter)); Papilio spp. (e.g. P. dardanus (swallowtail butterfly)); Antheraea spp. (e.g. A. yamamai (japanese oak silkmoth); Trichoplusia spp. (e.g. T. ni (cabbage looper)); Callosobruchus spp. (e.g. C. maculatus (cowpea weevil)); Rhynchosciara spp. (e.g. R. Americana (fungus gnat)); Sphaerius spp. (minute bog beatle); Ixodes spp. (e.g. I. scapularis (black-legged tick)); Diaphorina spp. (e.g. D. citri (asian citrus psyllid)); Meladema spp. (e.g. M. coriacea (Black Predacious Diving Beetle); Rhipicephalus spp. (e.g. R. appendiculatus (brown ear tick)); Amblyomma spp. (e.g. A. americanum (lone star tick); Toxoptera spp. (e.g. T. citricida (brown citrus aphid); Hister spp.; Dysdera spp. (e.g. D. erythrina (cell spider)), Lonomia spp. (e.g. L. obliqua (caterpillar)); and Culex spp. (e.g. C. pipiens (house mosquito)): and

(2) an insect that causes unwanted damage to substrates or materials, such as insects that attack plants, wood, seeds (e.g., stored seeds), grain (e.g., stored grain), manmade structures, etc. Insect examples of such pests include household insects, ecto-parasites and insects and/or arachnids such as, by way of example and not limitation, flies, spider mites, thrips, ticks, red poultry mite, ants, cockroaches, termites, head and body lice, crickets including house-crickets, silverfish, booklice, beetles, earwigs, mosquitoes and fleas.

In some embodiments, the insects can be a plant-eating phytophagous insect, such as Collembola, Isoptera, Coleoptera, Diptera, Hymenoptera, Lepidoptera, Orthoptera, Hemiptera, Thysanoptera insects or agricultural pests. Specific example includes long-winged tortrix moth species, genus of Adoxophyes, clearwing, cutworm, Cotton leaf corrugated armyworm, Anticarsia gemmatalis, archips, Argyrotaenia, Noctua, Busseolafusca, Pink spotted frog, Carposina sasakii, frog, Choristoneura, Clysia ambiguella, leaf rollers frog, Cnephasia, Coleophora, Thaumatotibia leucotreta, leaf roller, Corn frog, Leaf pine needles Moth, diamond, pink frog, eucosma, Euproctis flava Bremer, cutworm, leguminivora, hedya, Noctua, Choi frog, Hyphantria cunea, Keiferia lycopersicella Walsingham, Leucoptera scitella, conopobathr, Lymantria, phyllocnist, Malacosoma, Mamestra brassicae, Manduca sexta, Apochemia, European corn frog, Pammene, Panolis, Pink bollworm, cotton bollworm, Pieris rapae Phthorimaea operculella, Pieris, diamondback moth, ermine moth, white wild frog, big frog, Zeiraphera acanthocolla, Paranthrene, Argyrotaenia, Tortrix, cabbage looper, tree ermine moth, Elateroidea, snout beetle, Atomaria linearis, beet stem flea beetle, Sitophilus, real image genus, Dermestes, LeptispaBaly, Coccinella, Leptinotarsa decemlineata, Echinocnemus squameus Billberg, Melolontha, tenebroides, Cleoninae, Anomala exoleta Faldermann altica, Bostrichus, scarab: Sitophilus oryzae, sitotroga, Tenebrionini, Tribolium, Trogoderma, genus beetles, flour beetlegenus, Genus Gryllotalpa, the case of the beetle, flea beetle genus, genus non-Blatta Blatta, Leucophaea maderae, Locusts, Periplaneta, grasshoppers, termites, thrips, Thrips, single thrips, Thrips palmi, Thrips, Scirtothrips aurantii Faure. Generally said insects are harmful to plants.

The term “insect” encompasses insects of all types and at all stages of development, including egg, larval or nymphal, pupal, and adult stages.

### Pesticides

“Pesticide” as used herein includes insecticides, herbicides, and fungicides. Likewise, the methods provided can include a step of contacting the pest, soil, plant, wood, seeds (e.g., stored seeds), grain (e.g., stored grain) or manmade structure with a pesticide and one or more inhibitor agents as discussed above (or the plant may be a transgenic plant expressing, for RNAi that interferes with one or more insect genes).

The pesticide can be any of the pesticides known in the art. Pesticides in which there is increased susceptibility and/or toxicity and/or reduced resistance to when using the compositions of the inventions include, but are not limited to, pesticide classes of synthetic pyrethroids, pyrethrum, organochlorines, organophosphates, carbamates, fomidines, organosulfurs and organotins, neonicotinoids, and/or spinosins.

An insecticide is a pesticide used against insects, which include ovicides and larvicides used against the eggs and larvae of insects, respectively. Insecticides include, but are not limited to: (i) organochlorine/organochloride compounds (e.g. Aldrin, Chlordane, Chlordecone, DDT, Dieldrin, Endosulfan, Endrin, Heptachlor, Hexachlorobenzene, Lindane (gamma-hexachlorocyclohexane), Methoxychlor, Mirex, Pentachlorophenol, TDE); (ii) organophosphate compounds (e.g. Acephate, Azinphos-methyl, Bensulide, Chlorethoxyfos, Chlorpyrifos, Chlorpyriphos-methyl, Diazinon, Dichlorvos (DDVP), Dicrotophos, Dimethoate, Disulfoton, Ethoprop, Fenamiphos, Fenitrothion, Fenthion, Fosthiazate, Malathion, Methamidophos, Methidathion, Mevinphos, Monocrotophos, Naled, Omethoate, Oxydemeton-methyl, Parathion, Parathion-methyl, Phorate, Phosalone, Phosmet, Phostebupirim, Phoxim, Pirimiphos-methyl, Profenofos, Terbufos, Tetrachlorvinphos, Tribufos, Trichlorfon and other (acetyl)cholinesterase binding agents; (iii) carbamate insecticide compounds (e.g. Aldicarb, Bendiocarb, Carbofuran, Carbaryl, Fenoxycarb, Methomyl, 2-(1-Methylpropyl)phenyl methylcarbamate; (iv) pyrethroids, Allethrin, Bifenthrin, Cyhalothrin, Lambda-cyhalothrin, Cypermethrin, Cyfluthrin, Deltamethrin, Etofenprox, Fenvalerate, Permethrin, Phenothrin, Prallethrin, Resmethrin, Tetramethrin, Tralomethrin, Transfluthrin; (v) neonicotinoids (e.g. Acetamiprid, Clothianidin, Imidacloprid, Nitenpyram, Nithiazine, Thiacloprid, Thiamethoxam and other synthetic analogues of nicotine; (vi) biological insecticides, e.g. plant-derived biological insecticides, such as, Anabasine, Anethole (e.g. for mosquito larvae), Annonin, Asimina (pawpaw tree seeds for lice), Azadirachtin, Caffeine, Carapa, Cinnamaldehyde (e.g. for mosquito larvae), Cinnamon leaf oil (e.g. for mosquito larvae), Cinnamyl acetate (e.g. for mosquito larvae), Deguelin, Derris, Derris (rotenone), Desmodium caudatum (leaves and roots), Eugenol (for mosquito larvae), Linalool, Myristicin, Neem (Azadirachtin), Nicotiana rustica (nicotine), eganum Harmala, Oregano oil (for Rhizopertha dominica beetle), Polyketide, Pyrethrum, Quassia, Tetranortriterpenoid, Thymol (e.g. for mites), and non-plant-derived biological insecticides, such as Bacillus thuringiensis (Bt toxin) and other biological insecticides, including products based on entomopathogenic fungi (e.g. Metarhizium anisopliae), nematodes (e.g. Steinemema feltiae) and viruses (e.g. Cydia pomonella granulovirus); and (vii) anti-feedants such as, for example, polygodial. Other insecticides are known in the art and are commercially available for example from agrichemical manufacturers such as Bayer CropScience AG (Monheim am Rhein, Germany), Syngenta (Basel, Switzerland), BASF (Ludwigshafen, Germany), Dow Agrosciences (Indianapolis, Ind.), Monsanto (St. Louis, Mo.), and/or DuPont (Wilmington, Del.).

### Compositions and Application Thereof

The pest, soil, plant, wood, seeds (e.g., stored seeds), grain (e.g., stored grain), or manmade structure can be contacted with the compounds or compositions (e.g., inhibitor and pesticide) provided herein in any suitable manner. For example, the pest, soil, plant, wood, seeds (e.g., stored seeds), grain (e.g., stored grain), or manmade structure can be contacted with the compounds or compositions in pure or substantially pure form, for example, an aqueous solution. In this embodiment, the pest, soil, plant, wood, seeds (e.g., stored seeds), grain (e.g., stored grain), or manmade structure may be simply “soaked” with an aqueous solution comprising the compound or composition. In a further embodiment, the pest, soil, plant, wood, seeds (e.g., stored seeds), grain (e.g., stored grain), or manmade structure can be contacted by spraying the pest, soil, plant, wood, seeds (e.g., stored seeds), grain (e.g., stored grain), or manmade structure with a liquid composition. Additional methods will be known to the skilled person.

Alternatively, the compounds or compositions provided may be linked to a food component of the pests in order to increase uptake of the compound or composition by the pest, such as in a bait.

The compounds or compositions provided may also be incorporated in the medium in which the pest grows in or on, on a material or substrate that is infested by the pest or impregnated in a substrate or material susceptible to infestation by the pest.

In another specific embodiment, the compounds or compositions can be used in a coating that can be applied to a substrate in order to protect the substrate from infestation by a pest and/or to prevent, arrest or reduce pest growth on the substrate and thereby prevent damage caused by the pest. In this embodiment, the composition can be used to protect any substrate or material that is susceptible to infestation by or damage caused by a pest, for example, substrates such as wood.

Any harvested plant can be attacked by insects. Flour beetles, grain weevils, meal moths and other stored product pests will feed on stored grain, cereals, pet food, powdered chocolate, and almost everything else in the kitchen pantry that is not protected. Larvae of moths eat clothes made from animal products, such as fur, silk and wool. Larvae of carpet beetles eat both animal and plant products, including leather, fur, cotton, stored grain, and even museum specimens. Book lice and silverfish are pests of libraries. These insects eat the starchy glue in the bindings of books. Other insects that have invaded houses include cockroaches which eat almost anything. Cockroaches are not known to be a specific transmitter of disease, but they contaminate food and have an unpleasant odor. They are very annoying, and many pest control companies are kept busy in attempts to control them. The most common cockroaches in houses, grocery stores, and restaurants include the German cockroach, American cockroach, Oriental cockroach, and brown banded cockroach.

The nature of the excipients and the physical form of the composition may vary depending upon the nature of the substrate that is desired to treat. For example, the composition may be a liquid that is brushed or sprayed onto or imprinted into the material or substrate to be treated, or a coating that is applied to the material or substrate to be treated. Provided herein are also methods for treating and/or preventing pest infestation on a substrate comprising applying an effective amount of any of the compositions described herein to said substrate.

In another embodiment, the compounds or compositions are used as a pesticide for a plant or for propagation or reproductive material of a plant, such as on seeds. As an example, the composition can be used as a pesticide or insecticide by spraying or applying it on plant tissue or spraying or mixing it on the soil before or after emergence of the plantlets.

Any of the compositions provided herein may be formulated to include the active ingredient(s) and all inert ingredients (such as solvents, diluents, and various adjuvants).

Spray adjuvants (additives) can be added to pesticides to enhance the performance or handling of those pesticides. Adjuvant may include surfactants, crop oils, antifoaming agents, stickers, and spreaders. Adjuvants may also include: surfactants (surface-active agent), such as emulsifiers (e.g. to disperse oil in water), wetting agents (e.g. to reduce interfacial tensions between normally repelling substances), stickers (e.g. to cause the pesticide to adhere to the plant foliage and also to resist wash-off), and spreader-stickers (e.g. combined products that provide better spray coverage and adhesion). Crop oils and crop oil concentrates are light, petroleum-based oils that contain surfactant. Antifoam agents (foam suppressants) may be used to suppress foam formed when pesticides are agitated in the spray tank.

Carriers may serve as the diluent for any of the formulations provided herein. The carrier is the material to which a formulated pesticide is added, e.g. for field applications. A carrier may be used to enable uniform distribution of a small amount of formulated pesticide to a large area. Carriers may include liquid, dry and foam carriers. Liquid carriers, e.g. for spray applications, may include water, liquid fertilizers, vegetable oils, and diesel oil. Dry carriers may be used to apply pesticides without further dilution and may include attapulgite, kaolinite, vermiculite, starch polymers, corn cob, and others. Dry fertilizers can also be carriers.

The compositions provided herein can be a sprayable formulation. Sprayable Formulations (with liquid carrier) include: water-soluble liquids (designated S or SL or SC: form true solutions when mixed with water); Water-soluble powders (designated SP or WSP: are finely divided solids that dissolve completely in water); emulsifiable concentrates (designated E or EC: are oil-soluble emulsifiers that form emulsions when mixed with water); wettable powders (designated W or WP: are finely ground solids consisting of a dry carrier (a finely ground hydrophilic clay), pesticide, and dispersing agents, form an unstable suspension when mixed with water); water-dispersible liquids (designated WDL, L, F, AS: are finely ground solids suspended in a liquid system and form suspension when added to water); water-dispersible granules (designated WDG or DF, also called dry flowables, are dry formulations of granular dimensions made up of finely divided solids that combine with suspending and dispersing agents). Sprayable formulations may be in the form of aerosols and may be applied as droplets.

The compositions provided herein can be a dry formulation. Dry Formulations (e.g. for direct application without dilution in a liquid carrier) include: granules (designated G: consist of dry material in which small, dry carrier particles of uniform size (e.g. clay, sand, vermiculite, or corn cob; with a granule size of e.g. less than 0.61 cubic inches) are impregnated with the active ingredient, and may be applied with granular applicators); pellets (designated P: are dry formulations of pesticide and other components in discrete particles usually larger than 0.61 cubic inches, and may be applied e.g. by hand from shaker cans or with hand spreaders for spot applications). Dry formulations may also be applied as a fine powder or dust. Or larger dry formulations, such as in baits.

In yet another embodiment, a method for treating and/or preventing insect growth and/or insect infestation of a plant or propagation or reproductive material of a plant, comprising applying an effective amount of any of the compounds or compositions herein described to a plant or to propagation or reproductive material of a plant.

The compounds or compositions provided may be in any suitable physical form for application to pests, to substrates, to cells, or administration to organisms susceptible to infestation or infected by pests.

In other embodiments, the compositions provided contain further excipients, diluents, or carriers.

The compositions of the invention can include various amounts of the compounds. For example, the compound can be present in an amount of between about 0.000001%-99% by weight of the composition (W/W), preferably 0.00001%-99% by weight (W/W), more preferably, 0.0001%-99% by weight (W/W), still more preferably 0.0002%-99% by weight (W/W). The referenced amounts can be applied or administered in one or more applications or doses given over time.

The methods of the invention can find practical applications in any area of technology where it is desirable to inhibit viability, growth, development, or reproduction of a pest. Particularly useful practical applications include, but are not limited to, (1) protecting plants against pest infestation; (2) protecting materials against damage caused by pests; and (3) generally any application wherein pests need to be controlled and/or wherein damage caused by pests needs to be reduced or prevented.

### Screen

Further provided is a method to screen for compounds that increase pesticide toxicity, for both pesticide resistant and susceptible insects.

### Kit

The invention also provides kits that include containers of the compounds or compositions described herein. It is contemplated that the compounds or compositions may be supplied as a “kit-of-parts” comprising the compound or subpart thereof in one container and an amount of a compound, subpart thereof, or a carrier in a second container and, optionally, one or more suitable diluents for the foregoing components in one or more separate containers. In these embodiments, the compounds, subparts, carriers, or other molecules may be supplied in a concentrated form, such as a concentrated aqueous solution. It may even be supplied in frozen form or in freeze-dried or lyophilized form. The latter may be more stable for long term storage and may be defrosted and/or reconstituted with a suitable diluent immediately prior to use.

In one aspect, a kit comprising a first container containing an inhibitor of resistance and/or an agent that increases susceptibility to a pesticide is provided. In one embodiment, the kit further comprises a second container. In one embodiment, the second container comprises a pesticide.

Containers, as used herein, includes receptacles of any shape or form that may be made of any suitable material, such as plastic, glass, metal, styrofoam, cardboard and the like, or any combination of such materials.

The kit may be supplied with suitable instructions for use. The instructions may be printed on suitable packaging in which the other components are supplied or may be provided as a separate entity, which may be in the form of a sheet or leaflet for example. The instructions may be rolled or folded for example when in a stored state and may then be unrolled and unfolded to direct use of the remaining components of the kit.

### EXAMPLES

### Example 1: Inhibiting Pesticide Resistance Through the Discovery of an “Achilles' Heel” Resistance Trait

Introduction

Insecticide/Pesticide resistance is an ongoing challenge for agricultural production and vector borne disease control. Development of chemical-based tools to combat/suppress resistance have been limited (e.g., piperonyl butoxide); however, Pittendrigh et al. (2008, 2014) proposed that “omics tools” could be used to identify “Achilles' heel” resistance traits (hereafter called Achilles' heel resistance traits); that is, resistance-related proteins that, when inhibited, would result in the reduction or loss of the pesticide resistance phenotype. Pittendrigh et al. (2008, 2014) predicted that such an approach would involve determining pathways that both contribute to resistance, but also, if inhibited can render the insect incapable of maintaining the resistance phenotype or, as a corollary of this concept, can make susceptible insects hyper-susceptible to pesticides.

The testing of the of the Achilles' heel resistance trait concept is logical in a model system, such as Drosophila melanogaster (hereafter referred to as Drosophila), which has been used over the last half century as a model organism to explore the mechanisms leading to insecticide resistance and the consequences of pesticide exposure. For example, the laboratory selected DDT-resistant 91-R strain was established over 60 years ago and has received intermittent DDT selective pressure over the aforementioned time interval (Merrell and Underhill 1956; Merrell 1960, 1965). Its counterpart population, 91-C, was derived from the same progenitor population as 91-R, however, it has not been exposed to DDT in the laboratory.

Previous studies have shown that DDT resistance in 91-R is related to physiological alterations (Strycharz et al., 2013), and multigenic mechanism involves stress response, cell survival, and neurological functions (Seong et al., 2017). Differential expression of cytochrome P450 monooxygenases (P450) (Brandt et al., 2002; Festucci-Buselli et al., 2005; Daborn et al., 2007; Seong et al. 2018), ATP Binding Cassette (ABC) transporters (Strycharz et al., 2013; Seong et al., 2016), and estrogen related receptor structural changes (Sun et al., 2015) have also been associated with the 91-R DDT resistance phenotype. However, work by Pedra et al. (2005), in Drosophila, demonstrated the role of energy-related pathways in the DDT-resistant phenotype (Pedra et al., 2005); pathways that are evolutionary conserved between insects and mammals and where inhibitors for some of the proteins are commercially available. Proposed herein is that the discovery of Achilles' heel resistance traits may lie in elucidating energy metabolism-related genes involved in the resistance phenotype, as energy metabolism is also key to the survival of an organism.

Since Stohlman and Lillie (1948) first observed the hyperglycemic effect of DDT, numerous studies have demonstrated an association between DDT and energy metabolism. Adult rodents exposed to high doses of DDT suffered defects in insulin secretion, glucose intolerance, and increased gluconeogenesis (Kacew and Singhal, 1974; Yau and Mennear, 1977). In humans, epidemiological studies have shown that DDT has been linked to the impairment of insulin secretory function and increases in the potential for the onset of diabetes (Everett et al., 2007; Cox et al., 2007; Lee et al., 2017). Perinatal DDT exposure reduces energy expenditure and may increase susceptibility to the metabolic dysfunction in female mice offspring (La Merrill et al., 2014). DDE (Dichlorodiphenyldichloroethylene), a primary metabolite of DDT, has also been associated with obesity, insulin resistance, and dysmetabolism (Lee et al., 2011).

As one of the key pathways in energy metabolism, the insulin/insulin-like growth factor (IGF)-like signaling (IIS) pathway is evolutionarily conserved across vertebrates and invertebrates; and inhibitors for some of the proteins in this pathway are commercially available. In Drosophila, the US pathway has been shown to play roles in growth and development (Underwood et al., 1994; Edgar, 2006; Engelman et al., 2006) and is also related to lifespan regulation, metabolism of xenobiotics, and stress resistance (Hwangbo et al., 2004; Saltiel and Kahn, 2001; Slack et al., 2011; Tatar et al., 2003).

Mutations of genes within the IIS pathway can alter lifespan and response to environmentally-induced stress, including response to oxidative stress, thermotolerance, ultraviolet radiation, heavy metal and hypoxia (Lithgow et al., 1995; Murakami and Johnson, 1996; Barsyte et al., 2001; Kenyon, 2005; Scott et al., 2002). The IIS pathway has also been implicated in changes in organismal responses to pesticides (Clancy et al. 2001; Li et al. 2016). How DDT affects the IIS pathway and the adaptations of this pathway following the evolution of xenobiotic resistance, however, are still unknown. Moreover, to the authors' knowledge, regulation and changes in the IIS pathway have not been investigated to date in DDT-resistant Drosophila strains.

In this example, (i) comprehensive transcriptomic and genomic analyses of the IIS insulin pathway using the highly DDT-resistant 91-R and DDT-susceptible 91-C strains/populations was performed, (ii) life-history parameters (lifespan, starvation resistance and carbohydrate homeostasis) between these two aforementioned strains was investigated, and (iii) these insights were used to identify and test for Achilles' heel resistance traits via inhibitors of IIS pathway proteins. Based on the results, this is the first presentation of the effects of regulation and mutations of genes associated with the IIS pathway in the highly DDT resistant strain 91-R. These insights provide a tangible strategy to use a known pair of inhibitors of IIS pathway proteins to test for the concept of an Achilles' heel resistance traits; herein is the first to describe that the use of one such inhibitor results in the loss of the resistance phenotype in highly DDT resistant insects and, additionally, it was observed that the inhibitor makes DDT-susceptible insects hyper-susceptible. It is the first time one was able to tangibly demonstrate the potential for genomics tools towards the discovery of Achilles' heel resistance traits in Drosophila, as first proposed by Pittendrigh et al. (2008, 2014). Additionally, this is the first demonstration that highly DDT resistant insects show dramatic alterations in their IS pathway, a pathway associated with insulin resistance. The potential for this strategy in future pest control approaches is discussed.

Materials and Methods

Drosophila melanogaster Strains

In the following study, the DDT resistant Drosophila strain 91-R and corresponding non-DDT selected control strain, 91-C, were established previously (Merrell 1960, 1965; Merrell and Underhill 1956; Seong et al., 2017, 2018), were used. These two fly strains were reared in separate colonies on a commercially available medium (Jazz-Mix Drosophila Food, Fischer Scientific, Cat. No. AS153) under the conditions of 26±1° C., 50% relative humidity and a 14 h light/10 h dark cycle. The 91-R strain has been continually exposed to DDT by maintaining the flies in a colony bottle in the presence of a 150 mg DDT/filter paper disk (Seong et al., 2018), and later selected in scintillation vials coated with DDT (Kim et al., 2018), while 91-C was maintained without any exposure to DDT. Flies were transferred to a bottle with fresh diet every three weeks.

Estimation of Differential Expression for IIS Insulin Signaling Pathway Gene

All RNA-seq read data sets were previously generated from 91-C and 91-R in triplicate (Seong et al., 2017) and, for this study, were retrieved from the National Center for Biotechnology Information (NCBI) Short Read Archive (SRA) database (accession number: SRX2611754-SRX2611759).

The software, CLC Genomics Workbench version 9.5 (Qiagen), was used to analyze the transcriptomic data. After removing Illumina adapters and filtered low quality reads, the trimmed data was obtained from the raw reads. Gene sequences of IS pathway were downloaded from the Flybase database (http://flybase.org) (Dos Santos et al., 2014) and then imported to CLC Genomics Workbench software as a reference gene set. The six sets of trimmed sequence reads were mapped to reference genome and realigned.

Expression levels of the IIS pathway genes were compared between 91-R and 91-C strains using the number of reads per kilobase per million (RPKM) of mapped RNA-seq reads. The false discovery rate (FDR) method was applied to determine the threshold p-values to differential expression and genes in insulin signaling pathway. A FDR of ≤0.05 and a log 2 fold-change of ≥1 were considered as the thresholds to determine significant differences in gene expressions.

Reverse Transcriptase-Quantitative PCR (RT-qPCR) Validation

RT-qPCR validation was carried out on insulin signaling pathway genes for validation of RNA-seq estimated differences between the 91-R and 91-C strains. Both the RT-qPCR method and first-strand cDNA synthesis were similar to that previously described by Seong et al. (2017). The RT-qPCR primers are shown in Primer Table and ribosomal protein 49 (rp49) was used as the reference gene. The reaction for RT-qPCR included 10 μL SYBR Select Master Mix (Applied Biosystems Inc., USA), 0.3 μM of each primer, approximately 100 ng cDNA, and sterilized water to a total volume of 20 μL. The thermocycler program was as follows: 95° C. for 3 min, 40 cycles of 95° C. for 10 s, 60° C. for 10 s, 72° C. for 20 s. All amplification reactions were performed with the StepOnePlus Real-Time PCR system (Applied Biosystems Inc., USA) with three biological replicates. The mean of the threshold cycle (Ct) values from the three replicates of each strain were determined and the relative expression levels calculated using the formula (2-ΔΔCt) (Livak and Schmittgen, 2001). Statistical analysis was performed using Student's t-test to compare the results between the two strains by using SPSS software (Version 19.0; SPSS Inc, USA).

PRIMER TABLE
Primers used for the qRT-PCR analysis

| Genes | Direction | Sequence (5′-3′) |
|---|---|---|
| qrl-PH | Forward | CTTTGGATTGGCTCGTATTGC |
| | | (SEQ ID NO: 1) |
| | Reverse | ATCGGGTAGCAACGTATTCTG |
| | | (SEQ ID NO: 21) |
| qPhKγ-PF | Forward | ATTCTCAACCAAATGCTGCG |
| | | (SEQ ID NO: 3) |
| | Reverse | CCGGATGGCCTCCTCAA |
| | | (SEQ ID NO: 4) |
| qHex-C-PA | Forward | GGCTATACTCAACGATACCACTG |
| | | (SEQ ID NO: 5) |
| | Reverse | CCACATTCCTCAACATCTTCAACG |
| | | (SEQ ID NO: 6) |
| qfbp-PF | Forward | CAGTAGCGGTGATTCCAAAATG |
| | | (SEQ ID NO: 7) |
| | Reverse | GGATGGAGTTGAGCAGCTG |
| | | (SEQ ID NO: 8) |
| qLpin-PL | Forward | ATCAACACCAAGGGCGAG |
| | | (SEQ ID NO: 9) |
| | Reverse | CGCTGAACAGTTGATCGACG |
| | | (SEQ ID NO: 10) |
| qACC-PA | Forward | AGATCCAAACCCGTCCAATC |
| | | (SEQ ID NO: 11) |
| | Reverse | GGAGTTGTGATTGGTGTTGC |
| | | (SEQ ID NO: 12) |
| qGlyS-PA | Forward | CCTCACATCCACAAATCCTCTG |
| | | (SEQ ID NO: 13) |
| | Reverse | GATACCCATCACCGTACACTC |
| | | (SEQ ID NO: 14) |
| qGly-P-PA | Forward | CCAAGACCTACCAGAACCAAG |
| | | (SEQ ID NO: 15) |
| | Reverse | GCTCCACTCCCCAAATCTC |
| | | (SEQ ID NO: 16) |
| qLpin-PE | Forward | GATGACGAAGCCCTCTATAACG |
| | | (SEQ ID NO: 17) |
| | Reverse | ATTATGAGGCCCACATCGG |
| | | (SEQ ID NO: 18) |
| qDilp6-PD | Forward | CCTCAGCGATGTGATACAGAAG |
| | | (SEQ ID NO: 19) |
| | Reverse | CAGCACAAATCGGTTACGTTC |
| | | (SEQ ID NO: 20) |
| qCCHa2-PA | Forward | ACTATGTGCGTCCGAATGG |
| | | (SEQ ID NO: 21) |
| | Reverse | GGGCGGGATAGTCATTAAAGTC |
| | | (SEQ ID NO: 22) |
| qDilp8-PB | Forward | GACAGAAGGTCCATCGAGTTC |
| | | (SEQ ID NO: 23) |
| | Reverse | TTCGGCTGATGTAGTGATGC |
| | | (SEQ ID NO: 24) |
| qGSK3β-PO | Forward | ATTCTTCAACCAAATGCTGCG |
| | | (SEQ ID NO: 25) |
| | Reverse | CCGGATGGCCTCCTCAA |
| | | (SEQ ID NO: 26) |
| qPEPCK-PA | Forward | CGCTTCCTCTCCATTCAGTATG |
| | | (SEQ ID NO: 27) |
| | Reverse | GCACTTTTCCACGTACTGTTTG |
| | | (SEQ ID NO: 28) |
| qFlo2-PJ | Forward | GTCATCTTCGTATAAGCAAACCG |
| | | (SEQ ID NO: 29) |
| | Reverse | ATCGTCTGCTTGATCTCCTG |
| | | (SEQ ID NO: 30) |

Amino Acid Sequence Comparisons of Insulin Signaling Pathway Between the 91-C and 91-R Strains

After separately mapping RNA-seq reads from 91-C and 91-R flies to the Drosophila insulin pathway reference genes, consensus sequences were obtained by using “Map Reads to Reference” tool of the CLC Genomic Workbench 9.5 (Qiagen) software package. Related mapping parameters were set as: minimum length fraction=0.9, minimum similarity fraction=0.8, insertion/deletion cost=3, and mismatch cost=3. The consensus sequences of all insulin pathway genes were translated from nucleic sequence to protein by using CLC Genomics Workbench. Sequence alignments of putative amino acid were performed with Geneious 11.0.2 software (http://www.geneious.com) (Kearse et al., 2012). The consensus base frequency of mapped reads compared to the reference sequence was generated as previously described by Seong et al. (2018). All mutations found from RNA-seq data were verified by PCR amplification.

Lifespan and Starvation Test

For lifespan assay, virgin females and males from the 91-R and 91-C strains were collected and reared separately on a commercially-available diet. To make sure the adults were in the same age, the new emerged fruit flies were collected from reared bottles which were emptied in five hours. Adults of the same sex were kept at a density of 30 per vial. Flies were transferred to fresh diet every four days. Mortality was monitored every two days. Six replicates were performed for each treatment.

Survival of 91-R and 91-C fruit flies was measured in vials with 8 ml 1% agar (starvation), which allowed flies access to water but not nutrition as previously described by Broughton et al. (2005). Each vial contained 20 flies of each sex were prepared for each strain. Mortality was recorded every four hours. For the starvation test, 3-4, 5-6 and 9-10-days old flies were used in each experiment. Three replicates were performed for each treatment.

For lifespan and starvation experiments, the median survival was calculated using the probit function in SPSS (Version 19.0; SPSS Inc, USA).

Longevity Assay at Different Levels of Sucrose in the Diet

Twenty virgin female and male flies from the 91-C and 91-R strains were collected and reared separately on blue diet (formula 4-24®, carolina.com) made with three different sucrose solutions in water: 5.13% sucrose for low sugar (LS), 17.1% for medium sugar MS and 34.2% for high sugar (HS). To prevent bacteria and mold growth, propionic acid (0.5%) and nipagin solution (1% Nipagin M (Tegosept M), p-hydroxybenzoic acid were added to all sucrose solutions used for making blue diets. There were four biological replicates for each treatment starting at different dates. Flies were provided with fresh diets every two weeks to prevent bacteria and mold growth and desiccation of diet. Mortality was recorded every two days.

Glycogen Assay

The glycogen contents of 91-R and 91-C female and male flies were measured at 0 h, 24 h, and 48 h, respectively, after starvation according to the method described in Tennessen et al. (2014). Data (ug/mg fresh weight) are expressed relative to the fresh body weight of each fly. Three replicates were performed for each treatment.

Inhibitors Treatment and DDT Bioassay

Flies of 1-2 day old (females and males) were introduced into a plastic vial (28 mm×95 mm height) and fed with inhibitors mixed in 5% sucrose solution. Inhibitors used were as follows: hydrazine sulphate (Hys; PEPCK inhibitor, Sigma-Aldrich, 10 mM), lithium chloride (LiCl; GSK3p inhibitor, Sigma-Aldrich, 20 mM) (Hussain et al., 2017; Mudher et al., 2004). Each inhibitor solution was socked on a cotton plug in a 1.5 ml centrifuge tube which was put in the plastic vial (Choi et al., 2017). After fed with inhibitor solutions for two days, 20 females or males from either the 91-R or 91-C strains were used in all DDT bioassays. To explore the effect of inhibitors, fruit flies under two different rearing conditions: optimal rearing condition and sub-optimal rearing condition were used in our study. Under optimal rearing condition, 100 pairs of flies were maintained in bottles (containing 50 ml standard medium) and allowed for laying eggs for four days. All adults were then removed to make sure the suitable density of larva. Differently, adults were not removed until emerging of next generation under sub-optimal rearing condition. Simply, optimal rearing condition provided enough food for flies, but sub-optimal rearing condition faced certain food pressure.

Mortality bioassays using DDT were conducted following the method of Strycharz et al. (2013). Briefly, various concentrations of DDT dissolved in acetone were transferred into 20 ml transparent glass vials and each rolled on its side in a fume hood till the acetone evaporated. Under optimal dietary rearing conditions, the DDT working concentration was serially diluted by 1-256 times from stock concentration (64000 μg/ml) for 91-R, and diluted by 40-400 times from stock concentration (800 μg/ml) for 91-C. Under sub-optimal dietary rearing conditions, the DDT working concentration was serially diluted by 2-1600 times from stock concentration (8000 mg/ml) for 91-R and 20-8000 times from stock concentration (400 mg/ml) for 91-C. After all the acetone evaporated, flies of different stains were placed into vials. Vials were capped with cotton plugs moistened with a 5% sucrose solution in distilled water. The number of dead flies were recorded after 24 hours.

Statistical Analyses.

For RT-qPCR, statistical analysis was performed using Student's t-test to compare the results between 91-C and 91-R. For glycogen assay and body weight, One-way ANOVA was used, and means comparisons were made by using Student's t test (p<0.05) by using SPSS software (Version 19.0; SPSS Inc., USA). The median lifespan under fed or starvation conditions were calculated using the Probit function in SPSS (Version 19.0; SPSS Inc, USA). For mortality bioassay, Probit analysis was conducted by using SPSS software (Version 19.0; SPSS Inc, USA).

Results

Differential gene expression in insulin signaling pathway between 91-C and 91-R.

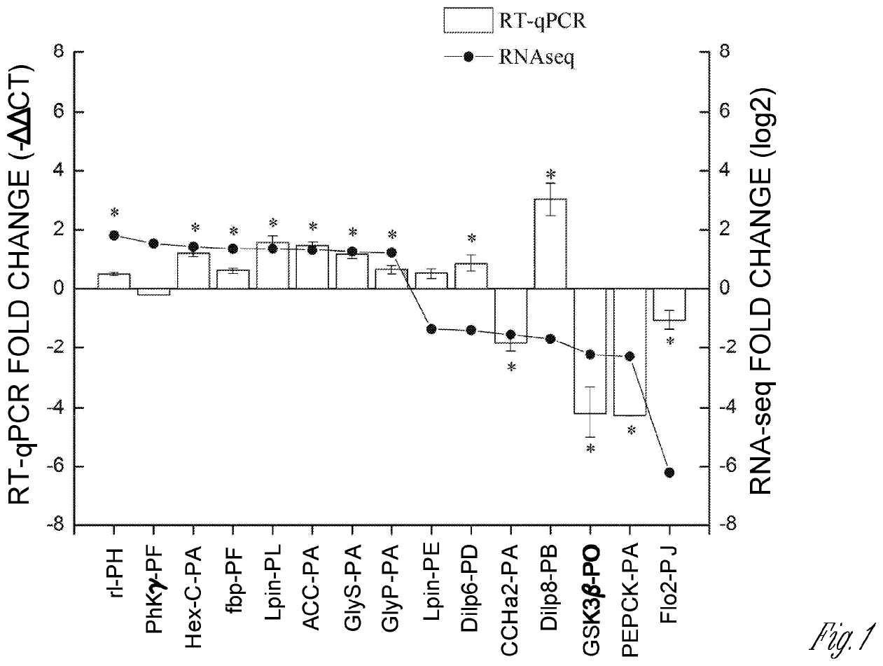

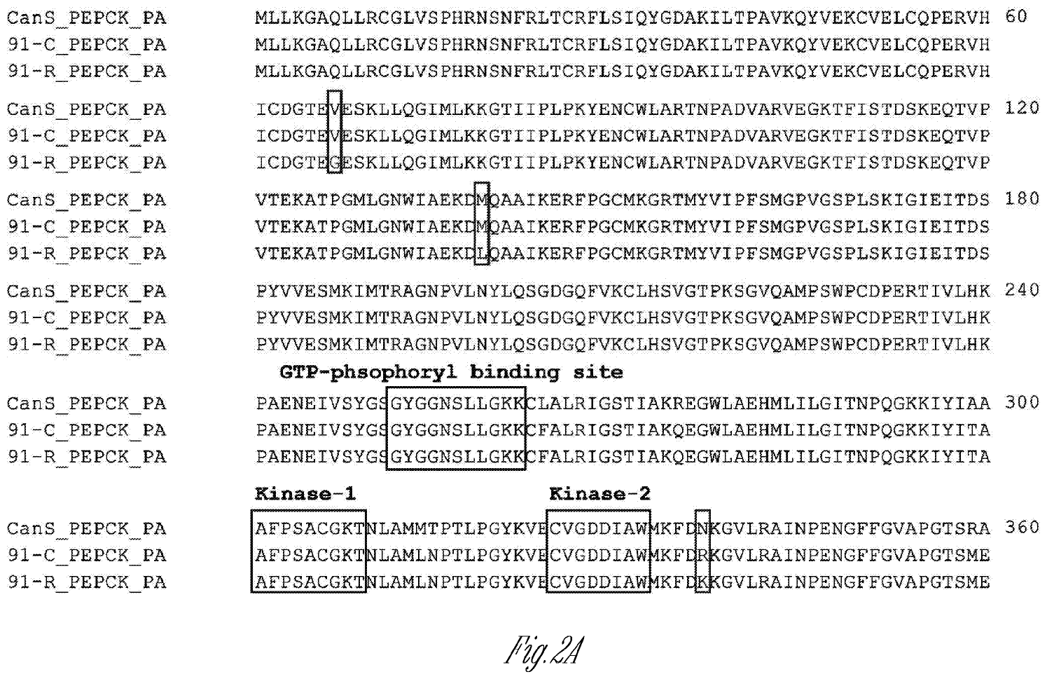

Fifteen candidate genes from the IIS insulin signaling pathway were predicted to be differentially-regulated via RNA-Seq; eight and seven genes were respectively up- and down-regulated in 91-R compared to 91-C(FDR<0.05 and log 2 fold change ≥|1.0|; Table 1, FIG. 1). These differentially-regulated candidate genes are listed below; eight up-regulated: rolled (rl-PH), Phosphorylase kinase gamma subunit (PhKγ-PF), Hexokinase (Hex-C-PA), Fructose-1,6-bisphosphatase (fbp-PF), Lipin (Lpin-PL), Acetyl-coa carboxylase/biotin carboxylase 1 (ACC-PA), Glycogen synthase (GlyS-PA), and Glycogen phosphorylase (GlyP-PA); and seven down-regulated: Lipin (Lpin-PE), Insulin-like peptide 6 (Dilp6-PD), Cchamide-2 (CCHa2-PA), Insulin-like peptide 8 (Dilp8-PB), Glycogen synthase kinase 3 beta (GSK3β-PO), Phosphoenolpyruvate carboxykinase (PEPCK-PA), and Flotillin (Flo2-PJ). Among these differentially regulated 15 genes predicted by RNA-seq, RT-qPCR validation of the predicted significant quantitative differences in expression levels among seven up-regulated and four down-regulated genes showed a similar trend between the results of RNA sequencing and RT-qPCR (r2=0.6793, p<0.01), confirming the predicted differential expression between strains based on Pearson's correlation coefficient test (FIG. 1).

TABLE 1
Annotations for differentially expressed transcripts of
insulin signaling pathway between the DDT-resistant 91-R
and-susceptible 91-C strains based on RNAseq analysis

| Gene | Functional | Annotation | Log₂ fold | |
|---|---|---|---|---|
| Symbol | annotation | Symbol | Change^{†} | FDR^{‡} |
| Flo2-PJ | Flotillin | CG32593 | −6.20 | 8.30E−04 |
| PEPCK-PA | Phosphoenolpyruvate | CG10924 | −2.30 | 0 |
| | carboxykinase 2 | | | |
| GSK3β-PO | Glycogen synthase | CG2621 | −2.74 | 1.51E−07 |
| | kinase 3 beta | | | |
| Dilp8-PB | Insulin-like peptide 8 | CG14059 | −1.68 | 1.80E−02 |
| CCHa2-PA | Cchamide-2 | CG14375 | −1.54 | 1.51E−04 |
| Dilp6-PD | Insulin-like peptide 6 | CG14049 | −1.39 | 2.05E−02 |
| Lpin-PE | Lipin | CG8709 | −1.35 | 5.15E−02 |
| GlyP-PA | Glycogen | CG7254 | 1.25 | 1.58E−02 |
| | phosphorylase | | | |
| GlyS-PA | Glycogen synthase | CG6904 | 1.27 | 2.84E−03 |
| ACC-PA | Acetyl-coa | CG11198 | 1.34 | 4.57E−03 |
| | carboxylase / biotin | | | |
| | carboxylase 1 | | | |
| Lpin-PL | Lipin | CG8709 | 1.37 | 1.44E−02 |
| fbp-PF | Fructose-1,6- | CG31692 | 1.38 | 1.11E−03 |
| | bisphosphatase I | | | |
| Hex-C-PA | Hexokinase C | CG8094 | 1.44 | 3.37E−05 |
| PhKγ-PF | Phosphorylase kinase | CG1830 | 1.55 | 9.95E−05 |
| | gamma subunit | | | |
| rl-PH | Mitogen-activated | CG12559 | 1.82 | 1.99E−02 |
| | protein kinase | | | |
| | (rolled) | | | |

^{†}Fold change was calculated as log2 91-C|91-R.
^{‡}FDR: False discovery rate. Differentially expressed genes were identified at threshold [FDR < 0.05 and log₂(fold change) ≥ |1.0|] of 91-C|91-R.

Nonsynonymous Variation of Differentially Regulated IIS Insulin Signaling Pathway Genes Between 914R and 91-C

Nucleotide variations associated with amino acid mutations in IIS insulin pathway genes were predicted from strain 91-R and 91-C (Table 2 and Table 2A). A total of 124 non-synonymous nucleotide changes were found among 36 of the 82 IIS insulin pathway genes open reading frames (ORFs) and no amino acid sequence difference was found in 46 out of the 82 IIS insulin pathway genes. Out of 124 nonsynonymous changes, 51.6% of mutations were fixed differently between 91-C and 91-R (homozygous), whereas the remaining 48.4% were segregating (unfixed; heterozygous) within both strains.

TABLE 2
Non-synonymous nucleotide and associated amino acid changes for differentially expressed
insulin signaling pathway genes in the DDT-resistant 91-R versus the −susceptible 91-C strains.

| | | | | | | | Amino | |
|---|---|---|---|---|---|---|---|---|
| | | CDS | SNP | Allelic variant (frequency) | | | acid | |
| Gene | Gene location | length | Position | 91-C | 91-R | Can-S | change | Fixation |

Under expressed genes

| | | | | | | | | |
|---|---|---|---|---|---|---|---|---|
| PEPCK-PA | 2R:18,527,430- | 1917 | 200 | T(1.0) | A(1.0) | T | V67G | Fixed |
| | 18,535,749 | | 415 | A(1.0) | T(1.0) | A | M139L | Fixed |
| | | | 1009 | C(0.80)/A(0.20) | A | A | R337K | Unfixed |
| | | | 1010 | G(0.80)/A(0.20) | A | A | | Unfixed |
| | | | 1612 | G(1.0) | A(0.84)/G(0.16) | G | E538K | Unfixed |
| | | | 1870 | G(1.0) | C(1.0) | G | A624P | Fixed |
| GSK3β-PO | X:2,633,952- | 1326 | 922 | A(1.0) | G(1.0) | A | T308A | Fixed |
| | 2,679,553 | | | | | | | |
| Lpin-PE | 2R:8,136,986- | 3138 | 778 | A(1.0) | C(1.0) | A | T260P | Fixed |
| | 8,156,654 | | 3134 | C(1 0) | T(1.0) | C | S1045F | Fixed |

Over expressed genes

| | | | | | | | | |
|---|---|---|---|---|---|---|---|---|
| Lpin-PL | 2R:8,136,986- | 2979 | 778 | A(1.0) | C(1.0) | A | T260P | Fixed |
| | 8,156,654 | | | | | | | |
| Hex-C | 2R:15,218,837- | 1365 | 1049 | A(1.0) | G(1.0) | A | K350R | Fixed |
| | 15,220,329 | | | | | | | |
| ERK (rl)-PH | 2R:1,071,462- | 800 | 433 | G(1.0) | G(0.73)/T(0.27) | G | G210* | Unfixed |
| | 1,125,927 | | | | | | | |

TABLE 2A
Non-synonymous nucleotide and associated amino acid changes for not differentially expressed insulin signaling
pathway genes in the DDT-resistant 91-R versus the —susceptible 91-C strains.

| | | | | | | | Amino | |
|---|---|---|---|---|---|---|---|---|
| | | CDS | SNP | Allelic variant (frequency) | | | acid | |
| Gene | Gene location | length | Position | 91-C | 91-R | Can-S | change | Fixation |
| Sdr-PA | 3R:14,916,628- | 2,607 | 28 | A(1.0) | C(1.0) | C | T10P | Fixed |
| | 14,925,083 | | 63 | T(1.0) | G(1.0) | G | F21L | Fixed |
| | | | 234 | C(1.0) | A(0.36)/C(0.64) | C | N79H | Unfixed |
| | | | 235 | A(1.0) | A(0.64)/C(0.36) | A | | Unfixed |
| | | | 359 | G(1.0) | A(1.0) | A | R120Q | Fixed |
| | | | 391 | G(1.0) | A(0.21)/G(0.79) | G | V131I | Unfixed |
| | | | 406 | A(1.0) | A(0.73)/G(0.27) | A | N136D | Unfixed |
| | | | 764 | C(1.0) | A(0.70)/C(0.30) | A | S255Y | Unfixed |
| | | | 841 | T(1.0) | A(0.68)/T(0.32) | T | L281I | Unfixed |
| | | | 1966 | C(1.0) | G(1.0) | C | Q656E | Fixed |
| | | | 2005 | G(1.0) | C(0.74)/G(0.26) | G | G669R | Unfixed |
| | | | 2151 | C(1.0) | T(1.0) | T | S718N | Fixed |
| sNPF-PB | 2L:20,027,915- | 846 | 439 | A(1.0) | T(1.0) | A | T147S | Fixed |
| | 20,056,373 | | 448 | G(1.0) | A(1.0) | G | D150N | Fixed |
| | | | 697 | A(1.0) | G(1.0) | A | T233A | Fixed |
| TkR99D-PC | 3R:29,950,796- | 1710 | 71 | A(1.0) | G(1.0) | G | S24N | Fixed |
| | 29,969,447 | | 1058 | C(0.25)/T(0.75) | T(1.0) | T | T352I | Unfixed |
| InR-PA | 3R:21,570,248- | 6435 | 573 | G(1.0) | T(0.73)/G(0.26) | G | Q191H | Unfixed |
| | 21,619,321 | | 587 | A(1.0) | A(0.73)/G(0.26) | A | H196R | Unfixed |
| | | | 590 | A(1.0) | T(0.60)/A(0.40) | A | H197L | Unfixed |
| | | | 1502 | A(1.0) | C(1.0) | C | N501T | Fixed |
| | | | 3448 | T(1.0) | C(1.0) | C | Y1150H | Fixed |
| | | | 5296 | C(1.0) | C(0.73)/G(0.26) | C | P1766A | Unfixed |
| | | | 6198 | C(1.0) | C(0.24)/G(0.76) | C | H2066Q | Unfixed |
| Fak-PB | 2R:19,430,659- | | 1363 | T(1.0) | G(1.0) | G | S455A | Fixed |
| | 19,437,259 | 3603 | 3307 | A(1.0) | G(1.0) | G | T1103A | Fixed |
| | | | 3442 | G(1.0) | A(1.0) | A | G1148S | Fixed |
| | | | 3457 | G(1.0) | A(1.0) | G | G1153S | Fixed |
| Fak-PD | 2R:19,430,659- | 4509 | 1363 | T(1.0) | G(1.0) | G | S455A | Fixed |
| | 19,437,259 | | 4213 | A(1.0) | G(1.0) | G | T1405A | Fixed |
| | | | 4348 | G(1.0) | A(1.0) | A | G1450S | Fixed |
| | | | 4363 | G(1.0) | A(1.0) | G | G1455S | Fixed |
| Lnk | 3R:25,888,542- | 2172 | 172 | G(1.0) | A(0.75)/G(0. 2) | G | V58M | Unfixed |
| | 25,893,270 | | 2039 | C(1.0) | A(0.74)/C(0.26 ) | A | P680Q | Unfixed |
| Nlaz-PB | 2L:1,359,837- | 738 | 63 | G(1.0) | T(1.0) | G | W21C | Fixed |
| | 1,361,821 | | 431 | C(1.0) | T(1.0) | C | T144I | Fixed |
| | | | 569 | G(1.0) | A(1.0) | A | S190N | Fixed |
| | | | 604 | A(1.0) | G(1.0) | G | T203A | Fixed |
| | | | 606 | T(1.0) | C(1.0) | C | | Fixed |
| melt-PB | 3-L:7,134,516- | 2967 | 902 | G(1.0) | C(0.22)/G(0.28) | G | S301T | Unfixed |
| | 7,143,074 | | 1271 | C(1.0) | C(0.71)/T(0.28) | C | T424M | Unfixed |
| | | | 2943 | A(1.0) | A(0.74)/G(0.26) | C | I981M | Unfixed |
| melt-PD | 3L:7,134,516- | 2979 | 1265 | C(1.0) | C(0.69)/T(0.31) | C | T422M | Unfixed |
| | 7,143,074 | | 2955 | A(1.0) | A(0.80)/G(0.20) | A | I985M | Unfixed |
| Pdk1-PD | 3L; 129,450- | 2511 | 1312 | G(1.0) | C(0.28)/G(0.72) | G | E438Q | Unfixed |
| | 145,895 | | | | | | | |
| step-PC | 2L:21 ;740,041- | 2184 | 821 | A(1.0) | G(1.0) | G | E274G | Fixed |
| | 21,757,466 | | | | | | | |
| Tsc1-PA | 3R:24,130,307- | 3303 | 1390 | G(1.0) | A(0.70)/G(0.30) | A | T464A | Unfixed |
| | 24,134,654 | | 1674 | G(1.0) | C(0.27)/G(0.73) | G | E558D | Unfixed |
| | | | 2315 | C(1.0) | G(1.0) | G | T772R | Fixed |
| | | | 2330 | G(1.0) | A(1.0) | A | R777H | Fixed |
| | | | 2380 | A(1.0) | A(0.64)/G(0.36) | A | T794A | Unfixed |
| unk-PA | 3R:23,149,653- | 1800 | 497 | C(1.0) | C(0.67)/G(0.33) | C | T166S | Unfixed |
| | 23,157,862 | | 498 | T(1.0) | C(1.0) | T | | Fixed |
| | | | 997 | A(1.0) | A(0.69)/(0.31) | A | T333A | Unfixed |
| Cbl-PB | 3L:8,424,954- | 2637 | 2383 | G(1.0) | G(0.39)/T(0.61) | G | A795S | Unfixed |
| | 8,432,246 | | 2392 | G(1.0) | G(0.37)/T(0 63) | G | A798S | Unfixed |
| Ptp61F-PA | 3L:1,342,493- | 1647 | 1420 | C(1.0) | C(0.80)/T(0.20) | C | P474S | Unfixed |
| | 1,475,257 | | | | | | | |
| Ptp61F-PC | 3L:1,242,493- | 1593 | 1080 | A(1.0) | A(0.72)/C(0.28) | A | K360N | Unfixed |
| | 1,475,257 | | 1183 | G(1.0) | A(0.28)/G(0.72) | G | A395T | Unfixed |
| | | | 1405 | C(1.0) | C(0.73)/T(0.27) | C | P469S | Unfixed |
| IKKβ-PB | 3R:16,046,347- | 2154 | 461 | G(1.0) | A(1.0) | A | G154D | Fixed |
| | 16,049,421 | | 1471 | G(1.0) | A(0.31)/G(0.68) | A | E491K | Unfixed |
| | | | 1511 | C(1.0) | A(0.35)/C(0.65) | C | T504K | Unfixed |
| SREBP-PA | 3L:19,793,935- | 3342 | 2120 | T(1.0) | G(0.27)/T(0.73) | T | L707R | Unfixed |
| | 19,798,979 | | | | | | | |
| Akt-PC | 3R:16,099,228- | 1836 | 149 | A(1.0) | A(0.74)/G(0.26) | A | N50S | Unfixed |
| | 16,105,670 | | 217 | A(1.0) | G(1.0) | G | T73A | Fixed |
| MNK (Lk6)-PA | 3R:11,750,931- | 3429 | 2170 | A(1.0) | A(0.29)/G(0.71) | A | K724X | Unfixed |
| | 11,764,457 | | 2171 | A(1.0) | A(0.72)/T(0.28) | A | | Unfixed |
| | | | 2294 | T(1.0) | A(0.69)/T(0.31) | T | L765Q | Unfixed |
| | | | 3287 | C(1.0) | C(0.33)/T(0.67) | C | A1096V | Unfixed |
| ERK (rl)-PG | 2R:1,071,462- | 996 | 628 | G(1.0) | G(0.83)/T(0.17) | G | G210* | Unfixed |
| | 1,125,928 | | | | | | | |
| TSC2 (gig)-PA | 3L:20,129,181- | 5544 | 2101 | C(1.0) | C(0.41)/T(0.59) | C | P701S | Unfixed |
| | 20,139,010 | | | | | | | |
| Rheb-PA | 3R:5,568,921- | 549 | 490 | A(1.0) | A(0.32)/T(0.68) | A | I164F | Unfixed |
| | 5,570,491 | | | | | | | |
| Raptor-PB | X:6,082,634- | 4866 | 269 | C(1.0) | T(1.0) | C | P90L | Fixed |
| | 6,090,201 | | 472 | C(1.0) | A(1.0) | C | L158M | Fixed |
| SOS-PA | 2L:13,813,816- | 4791 | 2105 | G(1.0) | A(1.0) | A | A702T | Fixed |
| | 13,819,824 | | | | | | | |
| Dfoxo-PG | 3R:14,056,945- | 1869 | 411 | A(1.0) | T(1.0) | T | E137D | Fixed |
| | 14,087,978 | | 938 | C(1.0) | A(1.0) | A | T313N | Fixed |
| | | | 1267 | C(1.0) | C(0.74)/T(0.26) | C | P423S | Unfixed |
| srl-PB | 3R:4,422,483- | 3,204 | 2591 | G(1.0) | A(0.31)/G(0.69) | G | R864H | Unfixed |
| | 4,429,335 | | | | | | | |
| srl-PD | 3R:4,422,483- | 3,177 | 2564 | G(1.0) | A(0.28)/G(0.72) | G | R855H | Unfixed |
| | 4,429,335 | | | | | | | |
| C3G-PI | X:6,780,790- | 4245 | 248 | C(0.60)/G(0.40) | G(1.0) | G | A83G | Unfixed |
| | 6,803,593 | | 2933 | G(1.0) | C(1.0) | G | A978P | Fixed |
| | | | 2964 | C(1.0) | T(1.0) | T | A988V | Fixed |
| Lar-PI | 2L:19,586,623- | 5826 | 64 | C(1.0) | T(1.0) | C | L22F | Fixed |
| | 19,732,069 | | | | | | | |
| GSK3β-PD | X:2,633,952- | 3204 | 2875 | A(1.0) | G(1.0) | A | T959A | Fixed |
| | 2,679,553 | | | | | | | |
| GSK3β-PG | X:2,633,952- | 1491 | 1162 | A(1.0) | G(1.0) | A | T388A | Fixed |
| | 2,679,553 | | | | | | | |
| GSK3β-PM | X:2,633,952- | 2319 | 320 | C(1.0) | G(1.0) | G | P107R | Fixed |
| | 2,679,553 | | 423 | T(1.0) | A(1.0) | A | D141E | Fixed |
| | | | 430 | T(1.0) | A(1.0) | A | S144T | Fixed |
| | | | 532 | A(1.0) | G(1.0) | G | T178A | Fixed |
| | | | 1810 | A(1.0) | G(1.0) | A | T603A | Fixed |
| GSK3β-PP | X:2,633,952- | 3381 | 119 | A(0.22)/C(0.78) | C(1.0) | C | N40T | Unfixed |
| | 2,679,553 | | 781 | C(1.0) | C(0.78)/G(0.19) | C | D261E | Unfixed |
| | | | 3053 | A(1.0) | G(1.0) | A | T1018A | Fixed |
| Exo70-PA | 3L:8,411,851- | 2083 | 1739 | A(1.0) | C(1.0) | C | N580T | Fixed |
| | 8,415,910 | | | | | | | |
| Hsl-PB | 2R:20,257,650- | 2646 | 904 | G(1.0) | C(1.0) | G | V302L | Fixed |
| | 20,261,726 | | 1153 | A(1.0) | C(1.0) | C | N385H | Fixed |
| | | | 2569 | C(1.0) | A(1.0) | A | H857T | Fixed |
| | | | 2570 | A(1.0) | C(1.0) | C | | |
| gig-PB | 3L:20,129,181- | 5535 | 2101 | C(1.0) | C(0.37)/T(0.63) | C | P701S | Unfixed |
| | 20,139,010 | | | | | | | |
| Myc-PB | X:3,373,159- | 2157 | 1106 | T(1.0) | A(0.27)/T(0.70) | A | I369N | Unfixed |
| | 3,391,697 | | | | | | | |

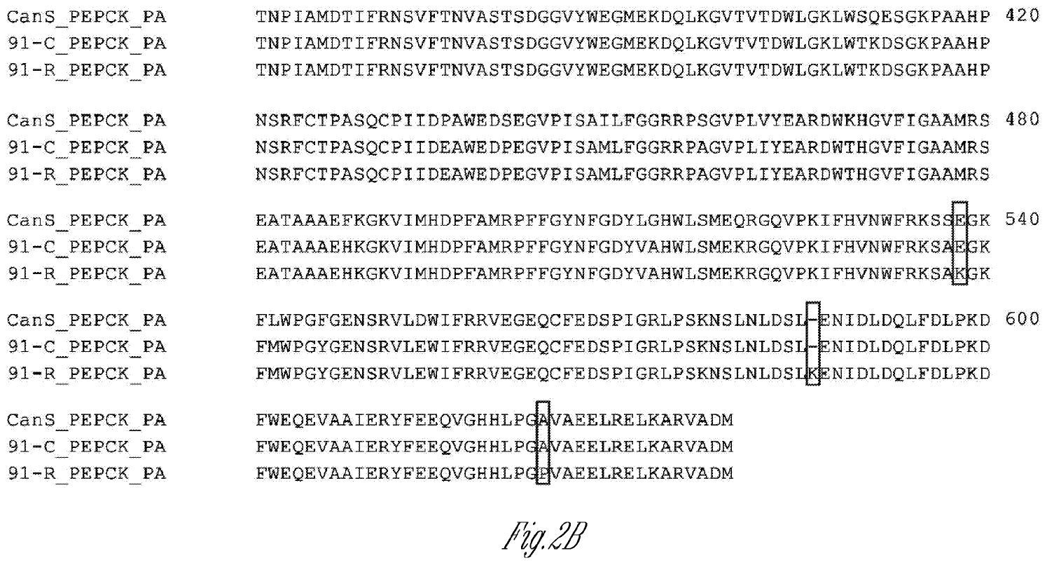

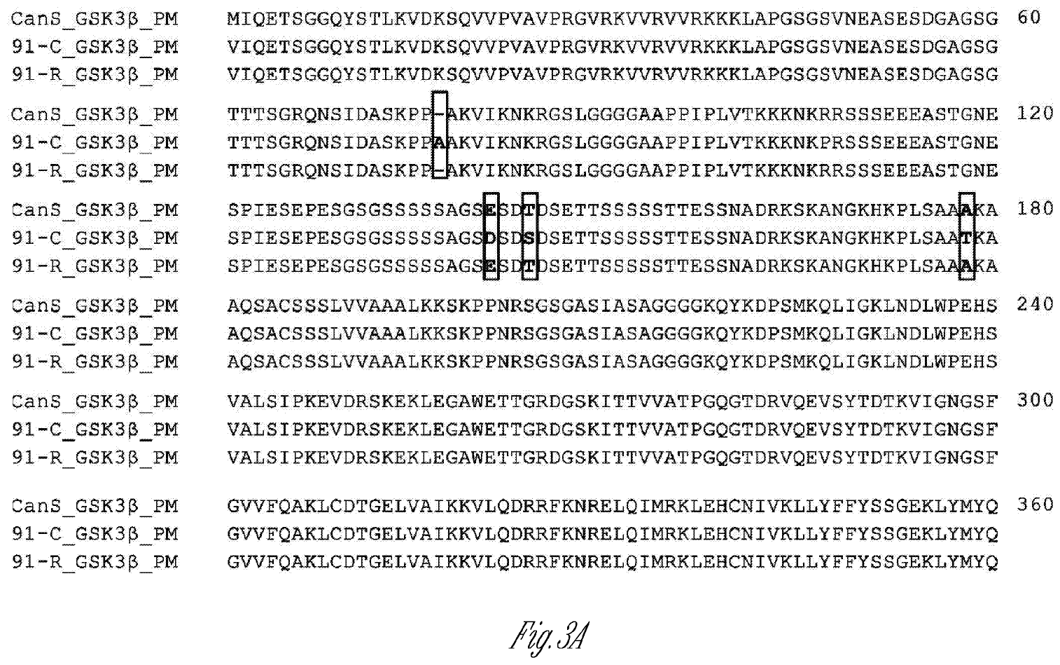

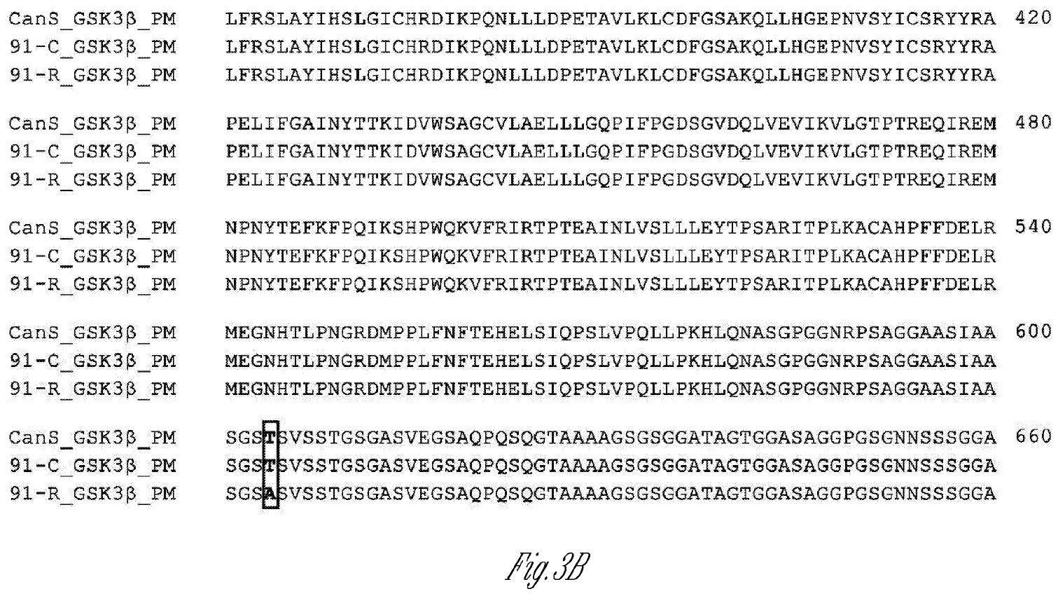

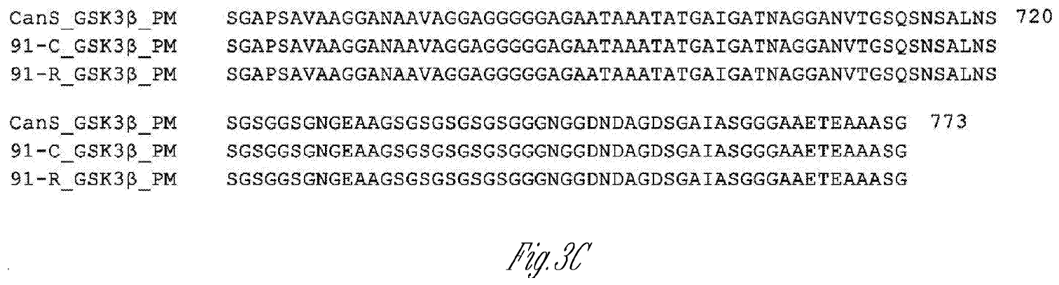

Among the fifteen differentially expressed genes, three up-regulated genes including Lpin-PL, Hex-C, and ERK (rl)-PH and three down-regulated genes, including PEPCK-PA, GSK3β-PO, and Lpin-PE showed non-synonymous mutations in 91-R. Among the significantly down-regulated transcripts in 91-R, for example, five non-synonymous mutations were found in PEPCK-PA at bp locations 200, 415, 1009, 1010 and 1870 and respectively led to amino acid changes V67G, M139L, R337K and A624P. Additionally, one fixed insertion was predicted at 1612 bp location led to amino acid change E538K in PEPCK-PA in 91-R (FIG. 2; Table 2). Only one mutation for GSK3β-PO (bp position 922, led to T308A amino acid change) and two mutations for Lpin-PE (bp position 788, and 3134, respectively led to amino acid changes T260P, and S1045F) were predicted and such mutations were defined as fixed (FIG. 3). On the other hand, for the three over-expressed genes (Lpin-PL, Hex-C, and ERK (rl)-PH), each gene was predicted to have one mutation between both strains. Each mutation was predicted at bp locations 778 for Lpin-PL (led to T260P amino change, fixed), 1049 for Hex-C(led to K350R amino change, fixed), and 443 for ERK (rl)-PH (introduce a premature stop codon, unfixed), respectively. Among not differentially expressed genes, 112 nucleotide non-synonymous changes were found (Table 2A). Out of these nonsynonymous changes, 50% of mutations were fixed differently, whereas 50% were unfixed between 91-C and 91-R. Moreover, six insertion/deletion polymorphisms were predicted and validated by Sanger sequencing in four genes only from the DDT-resistant 91-? strain (Table 3). Among them, five fixed mutations were found at bp location 320, 423, 430, 532, and 1810 in GSK3β-PM, respectively led to P107R, D141E, S144T, T178A, and T603A amino acid changes (Table 2A). When compared to 91-C, two insertions and four deletions were found from four genes in 91-R. Among these mutations, five were fixed and only one was unfixed (Table 3).

TABLE 3
Insertion/deletion polymorphisms predicted in the DDT-resistant 91-R strain.

| | | | | | | | | | Introduce | |
|---|---|---|---|---|---|---|---|---|---|---|
| | | | | Indel | | | | | a pre- | |
| | | CDS | | Position | | Allelic | | | mature | |
| | Gene | length | | (bp | | variant | | Type of | stop | Sequencing |
| Gene | location | (bp) | Strain | location) | Ins/Del | (frequency) | Fixation | mutation | codon | verification* |
| PEPCK- | 2R:18,527,430- | 1917 | 91-R | 1755- | Insertion | GAA(1.0) | Fixed | Frame | No | Yes |
| PA | 18,535,749 | | | 1757 | | | | shift | | |
| C3G-PI | X:6,780,790- | 4245 | 91-R | 254-265 | Deletion | GGTGGCG | Fixed | Frame | No | Yes |
| | 6,803,593 | | | | | GCAGT(1.0) | | shift | | |
| | | | | 1962 | Deletion | AGC(1.0) | Fixed | Frame | No | Yes |
| | | | | | | | | shift | | |
| GSK3β- | X:2,633,952- | 2319 | 91-R | 227-228 | Deletion | CCG(0.81)/gap | Unfixed | Frame | No | Yes |
| PM | 2,679,553 | | | | | | | shift | | |
| Myc-PB | X:3,373,159- | 2157 | 91-R | 1113- | Deletion | AACAAC(1.0) | Fixed | Frame | No | Yes |
| | 3,391,697 | | | 1117 | | | | shift | | |
| | | | | 1812- | Insertion | GCT(1.0) | Fixed | | | Yes |
| | | | | 1815 | | | | | | |

*Yes means normal sequencing showed same results with transcriptome sequencing.

Fitness Cost: Longevity and Starvation of 91-R and 91-C

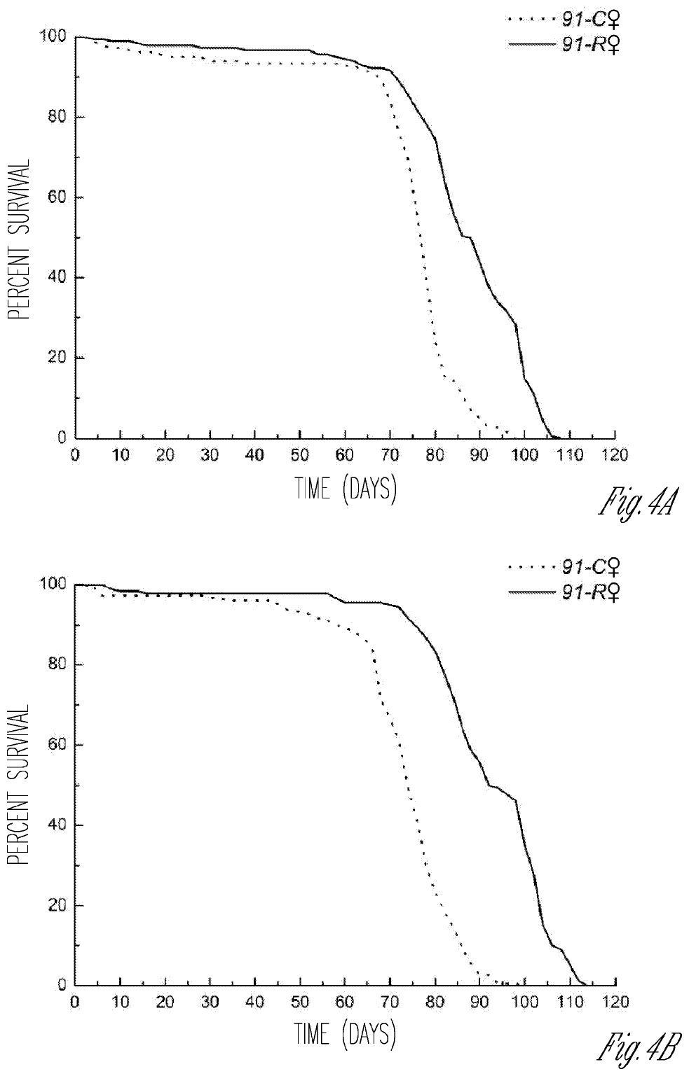

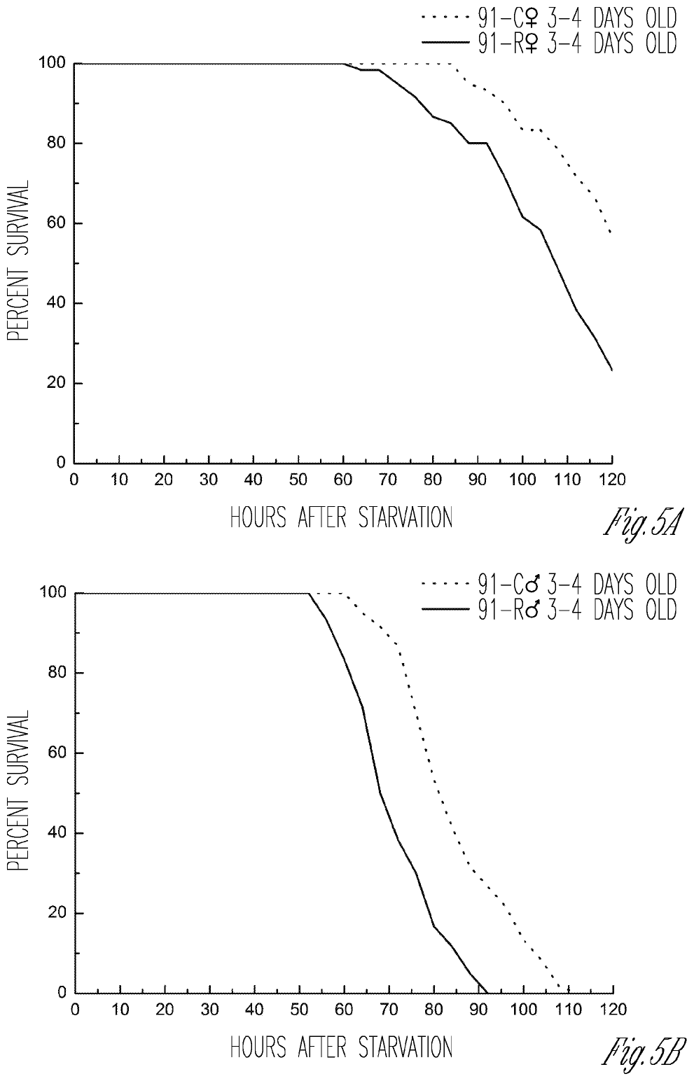

In order to investigate a fitness costs resulting from resistance to DDT, the longevity and starvation of female and male of 91-C and 91-R strains were assayed. The longest lifespan was 98 days and 108 days for 91-C and 91-R females (FIG. 4A), and 100 days and 114 days for 91-C and 91-R males (FIG. 4B), respectively. The median lifespan for female was found to be 85.29 (95% CI: 80.45-90.79) days for 91-R compared to 72.18 (95% CI: 67.36-77.34) days for 91-C. The median life expectancy for 91-C and 91-R males is 70.58 (95% CI: 64.45-77.12) and 91.80 (95% CI: 85.81-99.39) days, respectively. The DDT-resistant 91-R strain showed significantly longer lifespan compared to 91-C for both male and female (P<0.01).

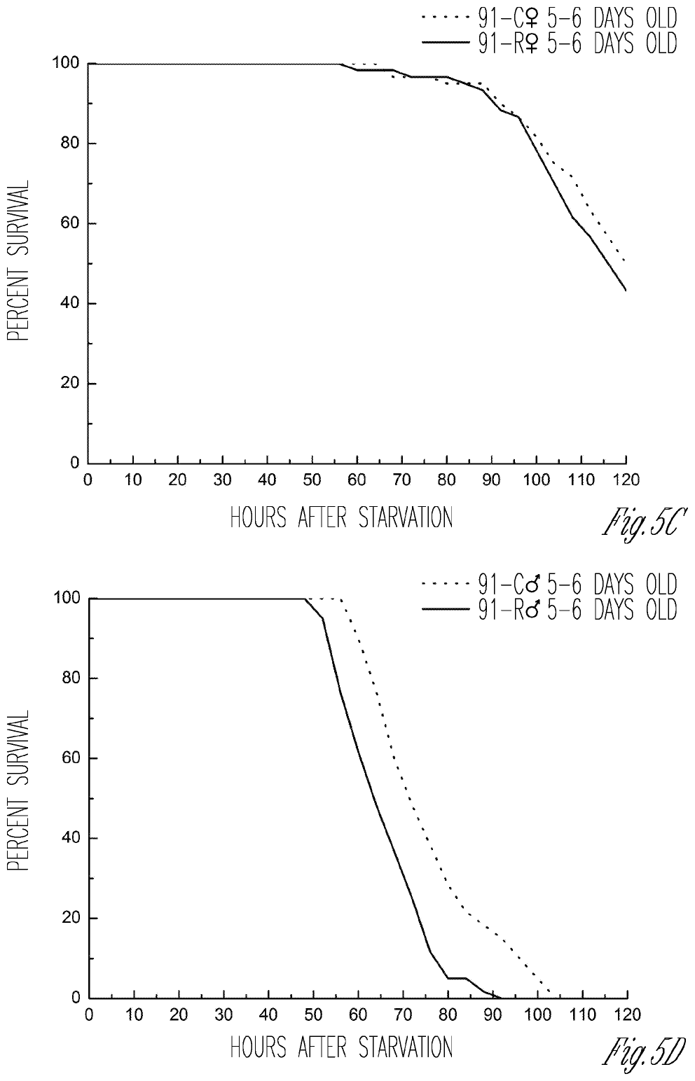

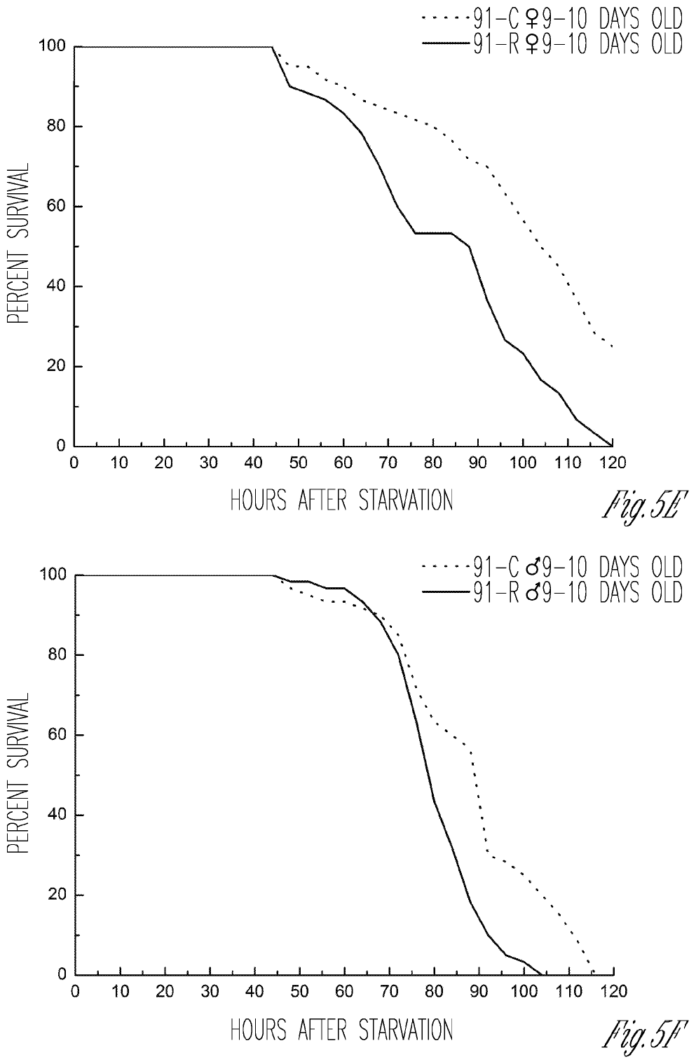

Three different age groups, 3-4, 5-6, and 9-10 days old, were examined to investigate survival rate of 91-C and 91-R flies after starvation. The median survival is 122.26 (95% CI: 119.52-125.76) and 106.37 (95% CI: 104.72-108.17) hours for 3-4 days old females of 91-C and 91-R, respectively. The median survival is 120.33 (95% CI: 117.51-123.79) and 116.33 (95% CI: 114.00-119.08) hours for 5-6 days old females of 91-C and 91-R, respectively. Males have a median survival of 83.85 (95% CI: 82.84-84.86) and 70.13 (95% CI: 69.23-71.03) hours for 3-4 days old 91-C and 91-R, respectively. The median survival is 74.83 (95% CI: 73.44-76.20) and 64.86 (95% CI: 63.96-65.76) hours for 5-6 days old males of 91-C and 91-I, respectively. In all cases, females survived longer than males at 3-4 and 5-6 days old. A tendency of survival reduction in the 91-R was observed as compared to 91-C when facing the starvation challenge (FIG. 5).

Glycogen Contents after Starvation

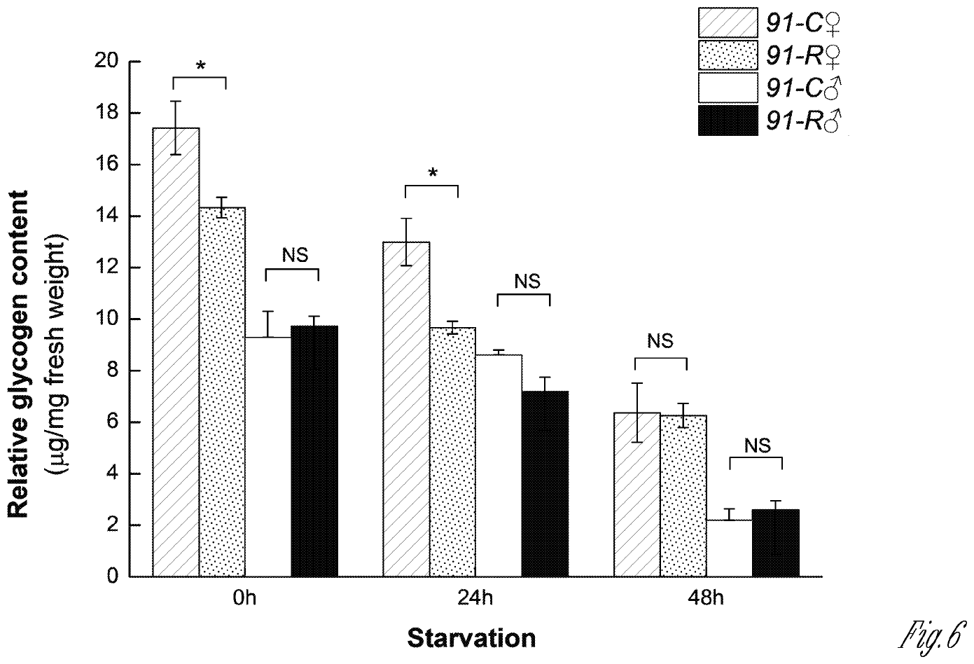

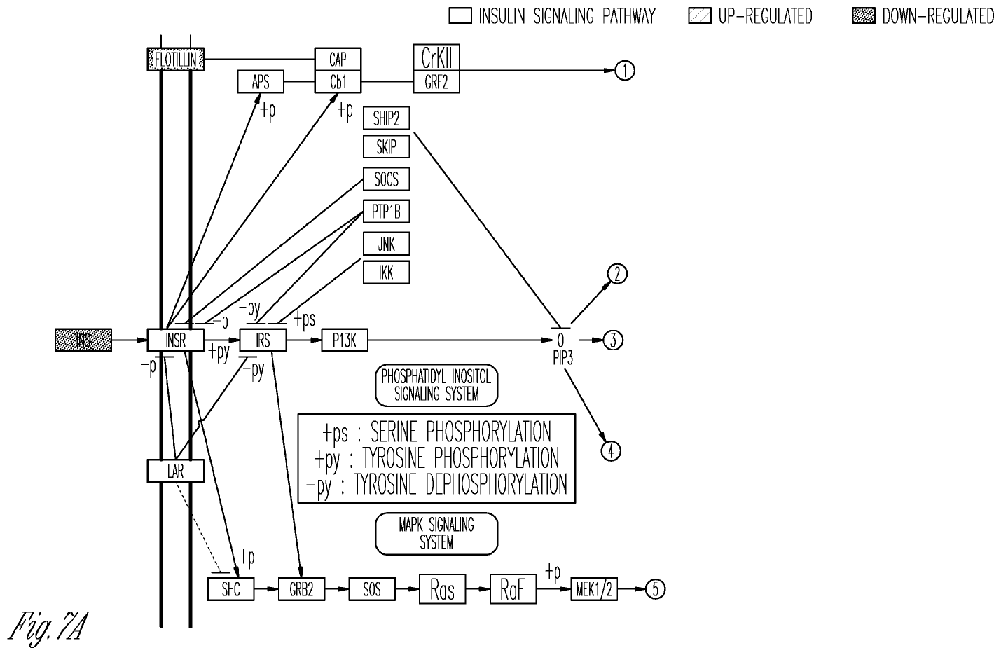

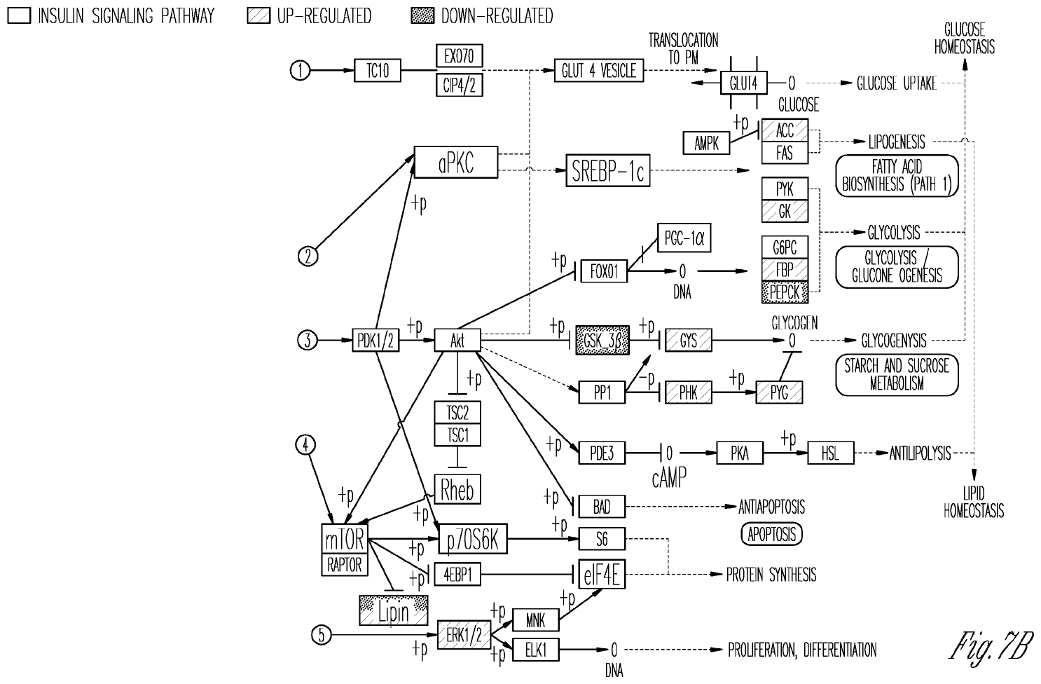

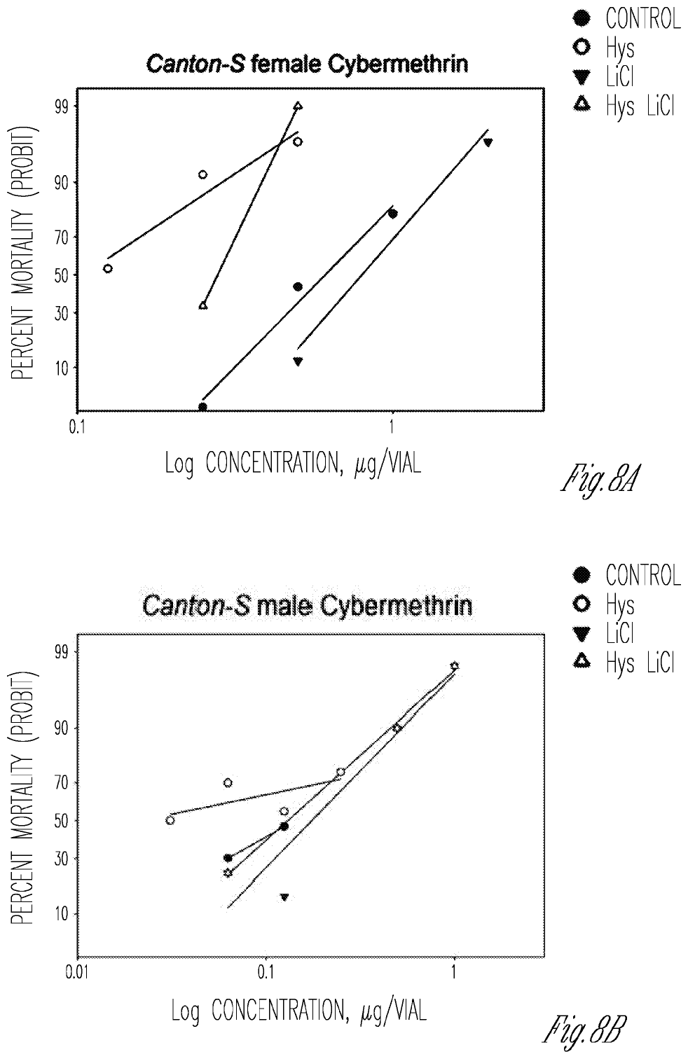

The glycogen contents before starvation (0 h) for 91-C and 91-R females were 17.41±1.03 and 14.33±0.40 μg/mg fresh weight, respectively (FIG. 6). After starvation for 24 hours, the average glycogen content for 91-C females decreased to 12.99 f 0.92 μg/mg fresh weight, which was significantly higher than 91-R females (9.66 f 0.25 μg/mg fresh weight). However, this difference in glycogen contents was not found at 48 hours starvation. For males, there was no significant difference between 91-C and 91-R at all three-time points.

Impact of Rearing Conditions on Adult Weights and LD₅₀ₛ

Flies under two different rearing conditions showed significantly different body weights for all genotypes and sexes (P<0.0001 for all groups) (Table 3A). Compared with flies under optimal rearing condition, flies facing food pressure (sub-optimal rearing condition) showed weight decreases. Rearing conditions also impact the LD₅₀ₛ of each of the genotypes (and their sexes) both in the presence and absence of dietary inhibitors (Tables 4A and 4B). Flies under optimal rearing condition were consistently more resistant/tolerant to DDT than sub-optimal rearing condition and in some cases these differences were dramatic. For example, the LD₅₀ of 91-C females under optimal rearing condition was 51.59 μg/vial and it decreased to 6.77 μg/vial under sub-optimal rearing condition (a ratio of 7.62). These differences were more dramatic for the 91-R DDT resistant strain. For females, an LD₅₀ of 136883.00 μg/vial was observed under optimal rearing conditions and 523.34 μg/vial for sub-optimal rearing conditions (a ratio of 261.56). For males, an LD₅₀ of 66073.00 μg/vial was observed under optimal rearing conditions and 143.70 μg/vial for sub-optimal rearing conditions (a ratio of 459.80).

TABLE 3A
Wet Weight of adult Drosophila
used in DDT bioassay (mn:, 20
adults, 3 days old)

| | Optimal rearing | Sub-optimal rearing |
|---|---|---|
| Fly-line | conditions*+ | conditions*+ |
| 91-C♀ | 23.50 ± 0.23 Aa | 17.60 ± 0.31 Ba |
| 91-C♂ | 16.63 ± 0.27 Ac | 12.37 ± 43.37 Bb |
| 91-R♀ | 23.23 ± 0.17 Aa | 17.73 ± 0.55 Ba |
| 91-R♂ | 17.57 ± 0.09 Ab | 12.63 ± 0.44 Bb |

*Comparisons between individual fly lines/genders under optimal and suboptimal rearing conditions whereby significant difference at P <0.0001 are given as A and B. For all fly lines and genders that differences in weights were highly significantly different between potimal and sub-optimal rearing conditions.
+Comparisons between different groups of flies (gender and fly lines) within a given rearing condition, whereby means are significantly different (at P <0.05) as given by a, b, or c.

TABLE 4A
LD₅₀'s of 91-C and 91-R in the presence of DDT without
(control) and with inhibitor feeding of Hydrazine sulfate
(Hys) or Lithium Chloride (LiCl) or both under optimal
dietary rearing conditions. See Table 3A for the representative
weights of adult Drosophila used in these bioassays

| | | LD₅₀ | (95% C.I.) | | Resistance |
|---|---|---|---|---|---|
| Strain | Inhibitors | (μg/vial) | lower | upper | Ratio |
| 91-C♀ | Control | 51.59 | 39.25 | 80.10 | — |
| | Hys | 9.78 | 7.93 | 11.87 | 5.28 |
| | LiCl | 12.17 | 11.16 | 13.14 | 4.24 |
| | Hys+LiCl | 5.26 | 3.54 | 7.37 | 9.81 |
| 91-C♂ | Control | 19.81 | 17.43 | 22.42 | — |
| | Hys | 4.99 | 3.97 | 6.07 | 3.97 |
| | LiCl | 7.22 | 6.47 | 8.01 | 2.74 |
| | Hys+LiCl | 4.16 | 3.79 | 4.56 | 4.76 |
| 91-R♀ | Control | 136883.00 | 86582.00 | 561499.00 | — |
| | Hys | 848.96 | 531.14 | 1235.00 | 161.24 |
| | LiCl | 83375.00 | 13558.00 | 1.27E+16 | 1.64 |
| | Hys+LiCl | 539.16 | 373.70 | 713.86 | 253.88 |
| 91-R♂ | Control | 66073.00 | 49796.00 | 118998.00 | — |
| | Hys | 302.86 | 206.49 | 397.35 | 218.16 |
| | LiCl | 3224.00 | 2305.00 | 5077.00 | 20.49 |
| | Hys+LiCl | 355.30 | 273.87 | 433.71 | 185.96 |

TABLE 4B
LD₅₀'s of 91-C and 91-R in the presence of DDT without
(control) and with inhibitor feeding of Hydrazine sulfate
(Hys) or Lithium Chloride (LiCl) or both under sub-optimal
dietary rearing conditions. See Table 3A for the representative
weights of adult Drosophila used in these bioassays.

| | | LD₅₀ | (95% C.I.) | | Resistance |
|---|---|---|---|---|---|
| Strain | Inhibitors | (μg/vial) | lower | upper | Ratio |
| 91-C♂ | Control | 6.77 | 5.00 | 9.96 | — |
| | Hys | 0.67 | 0.54 | 0.84 | 10.10 |
| | LiCl | 2.96 | 1.99 | 5.13 | 2.29 |
| | Hys+LiCl | 0.70 | 0.55 | 0.91 | 9.67 |
| 91-C♀ | Control | 2.12 | 1.50 | 3.41 | — |
| | Hys | 0.20 | 0.14 | 0.29 | 10.60 |
| | LiCl | 0.94 | 0.79 | 1.15 | 2.26 |
| | Hys+LiCl | 0.15 | 0.10 | 0.23 | 14.13 |
| 91-R♀ | Control | 523.34 | 256.00 | 941.84 | — |
| | Hys | 98.93 | 72.34 | 135.53 | 5.29 |
| | LiCl | 157.63 | 97.63 | 215.58 | 3.37 |
| | Hys+LiCl | 158.05 | 98.93 | 280.70 | 3.31 |
| 91-R♂ | Control | 143.70 | 84.66 | 209.98 | — |
| | Hys | 12,92 | 9.19 | 17.03 | 11.12 |
| | LiCl | 126.69 | 88.60 | 167.47 | 1.13 |
| | Hys+LiCl | 9.55 | 5.34 | 14.40 | 15.05 |

Longevity Assay at Different Levels of Sucrose in the Diet

The 91-C strain responded differently from that of 91-R flies when reared on sucrose-amended diet. The median lifespan of 91-C females was 49, 32 and 27 days when reared on low, medium and high sucrose diets, while 91-C males had a median lifespan of 26, 32, 23 days when reared on low, medium and high sucrose diets, respectively. Female 91-R flies fed with low, medium and high sugar had a median lifespan of 14, 19 and 16 days, indicating that 91-R female has a truncated lifespan when compared to either 91-C females or males at comparable sucrose levels (p<0.05). 91-R males had a median lifespan of 20, 24 and 24 days when reared on low, medium and high sucrose diets. At low and medium sucrose diets, 91-R males had a significantly truncated lifespan compared with 91-C flies (p<0.05).

Discovery an Achilles' Heel Resistance Trait:

The Effects of Hydrazine Sulphate and Lithium Chloride on DDT Sensitivity of 91-R and 91-C Strains

An oral feeding of the PEPCK and GSK3p inhibitors, hydrazine sulphate and lithium chloride, significantly decreased the LD₅₀ values of males and females for both 91-R and 91-C strains (Table 4). Under optimal rearing condition (Table 4A), Hys feeding reduced the level of DDT resistance in 91-R females by 161.2-fold, and in 91-R males by 218.2-fold. LiCl feeding reduced the level of DDT resistance in 91-R females and males by 1.6, and 20.5-fold respectively. For susceptible line 91-C, feeding with Hys reduced DDT resistance by 5.3 and 4.0-fold for females and males, respectively. Feeding with LiCl reduced DDT resistance by 4.2 and 2.7-fold for females and males, respectively. Flies of 91-R and 91-C strains became more sensitive for DDT after combing both Hys and LiCl together. Hys+LiCl feeding reduced DDT resistance level by 253.9 and 186-fold for 91-R females and males, respectively. The combine treatment decreased level of DDT resistance of 91-C females and males by 9.8 and 4.8-fold, respectively.

A similar trend was obtained under sub-optimal rearing condition (Table 4B). Feeding with Hys reduced DDT resistance by 10.1-10.6-fold in the 91-C strain, and 5.3-11.1-fold for 91-R, respectively. By comparison, LiCl decreased DDT resistance by 2.3-fold for 91-C females, 3.3-fold for 91-R females, and 2.3 for 91-C males (with no significant decreased in resistance levels for 91-R males). The combination of the two inhibitors was most effective on resistance levels in 91-R males (15.1-fold reduction).

Discussion

The analysis of the insulin signaling pathway, by comparing DDT resistant and susceptible Drosophila strains, resulted in the identification of Achilles' heel resistance traits (PEPCK and GSK3β) whereby a known inhibitor of PEPCK resulted in upwards of a 161.2-218.2 resistance ratio reduction in the DDT resistant flies (91-R) (under optimal rearing conditions) and resulted in hyper-susceptibility of the non-DDT selected flies (91-C) to DDT. In comparison, Lithium chloride, GSK3p inhibitor, decrease 1.6-20.5-fold of DDT resistance in 91-R. In the study, these two inhibitors: PEPCK inhibitor and GSK3β inhibitor, generally did not have an additive or synergistic effect of bringing down DDT resistance when combined feeding in both flies reared in optimal and sub-optimal rearing conditions.

Such Achilles' heel resistance traits were identified through determining the differences in the insulin signaling pathway between DDT resistant and susceptible Drosophila populations of common origin and then targeting a pair of candidate genes with known inhibitors for their protein products. Herein is the first report of the impact of the rearing conditions, and the weights of adult flies, on the LD₅₀s of the 91-R and 91-C strains. These results are in keeping with the work of Way (1954), which indicated that DDT was more toxic to the smaller Diataraxia oleracea L. larvae. The study of Buhler and Shanks (1970) also suggested that salmon with lower body weight had a lower lethal dose of DDT than the larger fish.

In the current study, it was found that multigenerational DDT exposure in a laboratory population of Drosophila resulted in changes in expression of transcripts and allelic diversity of genes associated with insulin signaling pathway. Specifically, eight and seven genes were up- and down-regulated, respectively, in 91-R strain. Additionally, a number of nonsynonymous nucleotide changes were present within and between the two strains, with the greatest number of non-synonymous nucleotide (associated amino acid changes), of differentially expressed genes, occurring in the PEPCK ORF. Moreover, the 91-R and 91-C strains showed differences in lifespan, glycogen levels (in females), and longevity differences both in the absence and presence of starvation conditions (respectively FIGS. 4 and 5), in keeping with known biological functions associated with genes contained within the insulin signaling pathway.

Previous work has shown that the insulin signaling pathway is impacted by pesticide exposure and plays an important role in xenobiotic metabolism and stress responses (Kenyon, 2005; Giannakou & Partridge, 2007). In this study, among down-regulated transcripts, flotillin is a constituent of lipid raft for glucose uptake (Hirabara et al., 2012) and flotillin's down-regulation is known to activate the IGF-1 receptor, ERK1/2 and Akt pathways (Jang et al., 2015). Li et al., (2016) observed that the IIS-related genes Akt, PI3K60, PI3K110, IRS, and PDK were reduced at 24 h but elevated 48 h after phoxim treatment in the silkworm midgut. Han et al. (2008) observed that Akt and mitogen-activated protein kinases (MAPK) were significantly activated by o,p′-DDT in macrophages.

Several of the other differentially expressed genes, which were observed in the study, have been previously associated with responses to pesticide exposure. For example, both Hexokinase C (Hex-C) and Fructose-1,6-bisphosphatase (FBP) were upregulated in 91-R. Previous work, with rat livers, has shown hexokinase activity was increased by 10% after sub-chronic exposure to malathion (Rezg et al., 2006). Hex-C phosphorylates hexoses (glucose) to glucose-6-phosphate, the start of either glycolysis or glycogen synthesis. Fructose-1,6-bisphosphatase (FBP) is upregulated in response to chronic DDT exposure; Kacew et al. (1972) indicated an increase in the FBP activity after DDT treatment in rat kidney cortex.

Under expression of PEPCK in this study suggest the hypothesis that gluconeogenesis in 91-R is/was lower than in 91-C. PEPCK is known to catalyze the first committed step in gluconeogenesis and plays an essential role in glucose metabolism (Burgess et al., 2007). PEPCK is overexpressed in all models of diabetes and is usually used as an indicator of gluconeogenic flux changes (Veneziale et al., 1983; Chakravarty et al., 2005); Glycogen synthase kinase 3 beta (GSK3β), one isoform of GSK3 which is linked to glycogen synthesis, also showed down-regulated in this study. Although there was no report indicated the direct relation of resistance and GSK3β, this multifunctional serine/threonine kinase has been shown to be affected by pesticides and causing pathogenesis of neurodegeneration (Songin et al., 2011; Kaytor and Orr, 2002; Martinez et al., 2002). GSK3β can also inhibit glycogen synthesis by suppressing Glycogen synthase (GlyS) through inhibitory phosphorylation (Lee et al., 2007). Down regulation of GSK30 and up regulation of GlyS in 91-R may accelerate glycogen synthesis. On the other side, Phosphorylase kinase gamma subunit (PhKγ), which activates glycogen phosphorylase (GlyP) to release glucose 1-phosphate from glycogen, was also upregulated in 91-R. In keeping with this previous work and the observations of the expression of the aforementioned genes, the glycogen assay indicated that 91-R females stored less energy in the form of glycogen than did the 91-C females (FIG. 6); no statistical differences were observed in males.

In addition to expression level changes between 91-C and 91-R, a number of nonsynonymous mutations were found in 91-R strain. Although many mutations were synonymous between the 91-C and 91-R, it was observed that 36 genes in the insulin pathway had nonsynonymous mutations, which may be associated with 91-R's long-term exposure to DDT. However, eight Insulin-like peptides (ILPs), which involved in controlling the growth of organisms, showed no nonsynonymous mutations in the study. The results implied that after chronically exposed to DDT, these ILPs are evolutionarily conserved in Drosophila (Brogiolo et al., 2001; Gronke et al., 2010). Among the differentially expressed genes, PEPCK has the greatest number of amino acid changes—both fixed and unfixed. Previous research has shown that a point mutation in PEPCK could result in upregulation of glucose-6-phosphatase and downregulation of glucokinase and GLUT2 (Burgess et al., 2007). Three fixed and three unfixed mutations were observed, as well as one insertion in the PEPCK-PA gene in 91-R. It remains to be determined if these mutations directly impact gluconeogenesis or the resistance phenotype in the 91-R strain.

As the enzyme regulating gluconeogenesis, PEPCK activity has been previously shown to be regulated by pesticides and in this study, it was both differentially expressed and had the greatest number of amino acid changes of any gene in the IIS pathway. Abdollahi et al. (2004) reported increased activity of PEPCK following sub chronic exposure to Malathion in rats. Diazinon and pentoxifylline could also significantly increase PEPCK activity on rat liver (Amirkabirian et al., 2007). In Drosophila, upregulation of PEPCK can be induced by xenobiotic damages and under starvation conditions (King-Jones et al., 2006; Zinke et al., 2002). Though the explicit reason for the downregulation of PEPCK is not known in this study, its involvement in DDT resistance was verified in inhibitor experiments.

Energy availability is the main limitation of organisms to survive under stressful conditions (Marron et al., 2003). In this study, 91-C females had higher glycogen content compared to 91-R females. The lower survival of 91-R after starvation could be attributed to the lower glycogen content. However, 91-R males did not show the same survival with 91-C males under starvation stress, though 91-R males and 91-C males showed similar initial glycogen content. Considering the similar physical activity level of both strains, the shorter survival time after starvation may demonstrate that 91-R has higher metabolic rates when facing stress. The metabolic rate is not simply because of regulation of gene expression in the IIS pathway (Hulbert et al., 2004). Further work is needed to explore the factors contribute to the difference and how 91-R regulate metabolic rates.

Previous work in C. elegans and Drosophila have demonstrated insulin signaling pathway regulates xenobiotic responses, development, life span, metabolism, and reproduction in females (Ikeya et al., 2002; Tatar et al., 2003). Afschar et al. (2015) observed that the mutant of the insulin receptor substrate chico exhibited increased resistance to DDT in Drosophila. McElwee et al. (2007) also observed that long-lived insulin signaling mutant showed overexpression of genes related to xenobiotic metabolism in C. elegans and Drosophila. In Drosophila, a mutant of the insulin-like receptor gene (InR) could be long-lived (Tatar et al., 2001). Mutations in daf-2, an insulin receptor-like gene result in prolonged lifespan and increase stress resistance in C. elegans (Kimura et al., 1997). In this study, 91-R showed 7 nonsynonymous mutations in InR_PA, including 2 fixed mutations. These mutations may be related to the long lifespan of 91-R. The results are also consistent with the research of Sub et al., (2008), which showed that two nonsynonymous mutations in the gene IGF1R are more enriched among long-lived humans. Insulin-like peptide-6 (Dilp6) expression from the fat body could extend Drosophila's lifespan (Bai et al., 2012). The results did not show any mutations for insulin-like peptide in 91-R. However, the qPCR results showed that Dilp6 was significantly up-regulated and our longevity studies showed longer lifespan of 91-R. It remains to be determine as to which, if any, molecular changes in the IIS pathway, in 91-R, cause the phenotypic changes in longevity both in the presence and absence of starvation.

### CONCLUSION

In conclusion, the data presented in this study indicate the differentially expressed transcripts and structural variations of insulin pathway genes of Drosophila between DDT-susceptible and -resistant strains. Within the differentially expressed genes, PEPCK has the greatest number of amino acid changes. PEPCK inhibitor, hydrazine sulphate, significantly reduce the DDT resistance of Drosophila. This decrease of DDT resistance occurred despite DDT-susceptible and -resistant populations, optimal and sub-optimal rear condition. This is the first report of the identity an Achilles' heel resistance trait in an insect population. Further work is needed to determine the suitability and practical application of PEPCK or GSK3β inhibitors in pest control across pesticide resistant lines, across pesticide classes, and across insect species.

### BIBLIOGRAPHY

Choi M Y, Tang S B, Ahn S J, et al. Effect of non-nutritive sugars to decrease the survivorship of spotted wing Drosophila, Drosophila suzukii. Journal of insect physiology, 2017, 99: 86-94.
Kim, J. H., Moreau, J. A., Zina, J. M., Mazgaeen, L., Yoon, K. S., Pittendrigh, B. R., & Clark, J. M. (2018). Identification and interaction of multiple genes resulting in DDT resistance in the 91-R strain of Drosophila melanogaster by RNAi approaches. Pesticide Biochemistry and Physiology.
Virkamaki, A., Ueki, K., & Kahn, C. R. (1999). Protein-protein interaction in insulin signaling and the molecular mechanisms of insulin resistance. The Journal of clinical investigation, 103(7), 931-943.
McElwee, J. J., Schuster, E., Blanc, E., Piper, M. D., Thomas, J. H., Patel, D. S., . . . & Gems, D. (2007). Evolutionary conservation of regulated longevity assurance mechanisms. Genome biology, 8(7), R132.
Songin, M., Ossowska, K., Kuter, K., & Strosznajder, J. B. (2011). Alteration of GSK-3β in the hippocampus and other brain structures after chronic paraquat administration in rats. Folia neuropathologica, 49(4), 319-327.
Amirkabirian, N., Teimouri, F., Esmaily, H., Mohammadirad, A., Aliahmadi, A., & Abdollahi, M. (2007). Protection by pentoxifylline of diazinon-induced toxic stress in rat liver and muscle. Toxicology mechanisms and methods, 17(4), 215-221.
Hulbert, A. J., Clancy, D. J., Mair, W., Braeckman, B. P., Gems, D., & Partridge, L. (2004). Metabolic rate is not reduced by dietary-restriction or by lowered insulin/IGF-1 signalling and is not correlated with individual lifespan in Drosophila melanogaster. Experimental gerontology, 39(8), 1137-1143.
Marron, M. T., Markow, T. A., Kain, K. J., & Gibbs, A. G. (2003). Effects of starvation and desiccation on energy metabolism in desert and mesic Drosophila. Journal of Insect Physiology, 49(3), 261-270.
Sun, W., Valero, M. C., Seong, K. M., Steele, L. D., Huang, I. T., Lee, C. H., . . . & Pittendrigh, B. R. (2015). A glycine insertion in the estrogen-related receptor (ERR) is associated with enhanced expression of three cytochrome P450 genes in transgenic Drosophila melanogaster. PLoS One, 10(3), e0118779.
Bai, H., Kang, P., & Tatar, M. (2012). Drosophila insulin-like peptide-6 (dilp6) expression from fat body extends lifespan and represses secretion of Drosophila insulin-like peptide-2 from the brain. Aging cell, 11(6), 978-985.
Kimura, K. D., Tissenbaum, H. A., Liu, Y., & Ruvkun, G. (1997). daf-2, an insulin receptor-like gene that regulates longevity and diapause in Caenorhabditis elegans. Science, 277(5328), 942-946.
Ikeya, T., Galic, M., Belawat, P., Nairz, K., & Hafen, E. (2002). Nutrient-dependent expression of insulin-like peptides from neuroendocrine cells in the CNS contributes to growth regulation in Drosophila. Current biology, 12(15), 1293-1300.
Grönke, S., Clarke, D. F., Broughton, S., Andrews, T. D., & Partridge, L. (2010). Molecular evolution and functional characterization of Drosophila insulin-like peptides. PLoS genetics, 6(2), e1000857.
Veneziale, C. M., Donofrio, J. C., & Nishimura, H. (1983). The concentration of P-enolpyruvate carboxykinase protein in murine tissues in diabetes of chemical and genetic origin. Journal of Biological Chemistry, 258(23), 14257-14262.
Suh, Y., Atzmon, G., Cho, M. O., Hwang, D., Liu, B., Leahy, D. J., . . . & Cohen, P. (2008). Functionally significant insulin-like growth factor I receptor mutations in centenarians. Proceedings of the National Academy of Sciences, 105(9), 3438-3442.
Burgess, S. C., He, T., Yan, Z., Lindner, J., Sherry, A. D., Malloy, C. R., . . . & Magnuson, M. A. (2007). Cytosolic phosphoenolpyruvate carboxykinase does not solely control the rate of hepatic gluconeogenesis in the intact mouse liver. Cell metabolism, 5(4), 313-320.
Hirabara, S. M., Gorjao, R., Vinolo, M. A., Rodrigues, A. C., Nachbar, R. T., & Curi, R. (2012). Molecular targets related to inflammation and insulin resistance and potential interventions. BioMed Research International, 2012.
Giannakou, M. E., & Partridge, L. (2007). Role of insulin-like signaling in Drosophila lifespan. Trends in biochemical sciences, 32(4), 180-188.
La Merrill, M., Karey, E., Moshier, E., Lindtner, C., La Frano, M. R., Newman, J. W., & Buettner, C. (2014). Perinatal exposure of mice to the pesticide DDT impairs energy expenditure and metabolism in adult female offspring. PLoS One, 9(7), e103337.
Han, E. H., Kim, J. Y., Kim, H. K., Hwang, Y. P., & Jeong, H. G. (2008). o, p′-DDT induces cyclooxygenase-2 gene expression in murine macrophages: Role of AP-1 and CRE promoter elements and PI3-kinase/Akt/MAPK signaling pathways. Toxicology and applied pharmacology, 233(2), 333-342.
Chakravarty, K., Cassuto, H., Reshef, L., & Hanson, R. W. (2005). Factors that control the tissue-specific transcription of the gene for phosphoenolpyruvate carboxykinase-C. Critical reviews in biochemistry and molecular biology, 40(3), 129-154.
Stohlman, E. F., & Lillie, R. D. (1948). The effect of DDT on the blood sugar and of glucose administration on the acute and chronic poisoning of DDT in rabbits. Journal of Pharmacology and Experimental Therapeutics, 93(3), 351-361.
Hodgson, E., & Levi, P. E. (1999). Interactions of piperonyl butoxide with cytochrome P450. In Piperonyl butoxide (pp. 41-II).
Brogiolo, W., Stocker, H., Ikeya, T., Rintelen, F., Fernandez, R., & Hafen, E. (2001). An evolutionarily conserved function of the Drosophila insulin receptor and insulin-like peptides in growth control. Current biology, 11(4), 213-221.
Kaytor M D, Orr H T. The GSK3 beta signaling cascade and neurodegenerative disease. Current Opinion in Neurobiology 2002; 12:275-8.
Martinez A, Castro A, Dorronsoro I, Alonso M. Glycogen synthase kinase 3 (GSK-3) inhibitors as new promising drugs for diabetes, neurodegeneration, cancer, and inflammation. Medicinal Research Reviews 2002; 22: 373-84
Pittendrigh, B. R., Margam, V. M., Sun, L., & Huesing, J. E. (2008). Resistance in the postgenomics age. Insect resistance management: Biology, economics and prediction, 39-68.
Pittendrigh, B. R., Margam, V. M., Walters, K. R., Steele, L. D., Olds, B. P., Sun, L., . . . & Clark, J. M. (2014). Understanding resistance and induced responses of insects to xenobiotics and insecticides in the age of “omics” and systems biology. In Insect Resistance Management (Second Edition) (pp. 55-98).
Kearse, M., Moir, R., Wilson, A., Stones-Havas, S., Cheung, M., Sturrock, S., . . . & Thierer, T. (2012). Geneious Basic: an integrated and extendable desktop software platform for the organization and analysis of sequence data. Bioinformatics, 28(12), 1647-1649.
dos Santos, G., Schroeder, A. J., Goodman, J. L., Strelets, V. B., Crosby, M. A., Thurmond, J., . . . & FlyBase Consortium. (2014). FlyBase: introduction of the Drosophila melanogaster Release 6 reference genome assembly and large-scale migration of genome annotations. Nucleic acids research, 43(D1), D690-D697.
Lee, J., & Kim, M. S. (2007). The role of GSK3 in glucose homeostasis and the development of insulin resistance. Diabetes research and clinical practice, 77(3), S49-S57.
Li, F., Hu, J., Tian, J., Xu, K., Ni, M., Wang, B., . . . & Li, B. (2016). Effects of phoxim on nutrient metabolism and insulin signaling pathway in silkworm midgut. Chemosphere, 146, 478-485.
King-Jones K, Homer M A, Lam G, Thummel C S (2006) The DHR96 nuclear receptor regulates xenobiotic responses in Drosophila. Cell Metab 4: 37-48.
Zinke I, Schutz C S, Katzenberger J D, Bauer M, Pankratz M J (2002) Nutrient control of gene expression in Drosophila: microarray analysis of starvation and sugar-dependent response. EMBO J 21: 6162-6173.
Abdollahi, M., Donyavi, M., Pournourmohammadi, S., & Saadat M. (2004). Hyperglycemia associated with increased hepatic glycogen phosphorylase and phosphoenol pyruvate carboxykinase in rats following sub-chronic exposure to malathion. Comparative Biochemistry & Physiology (C), 137, 343-347.
Kacew, S., Singhal, R. L., & Ling, G. M. (1972). DDT-induced stimulation of key gluconeogenic enzymes in rat kidney cortex. Canadian journal of biochemistry, 50(2), 225-229.
O'Brien, R. M., & Granner, D. K. (1990). PEPCK gene as model of inhibitory effects of insulin on gene transcription. Diabetes Care, 13(3), 327-339.
Slack C, Giannakou M E, Foley A, Goss M, Partridge L (2011) dFOXO-independent effects of reduced insulin-like signaling in Drosophila. Aging Cell 10(5):735-748.
Broughton, S. J., Piper, M. D., Ikeya, T., Bass, T. M., Jacobson, J., Driege, Y., . . . & Partridge, L. (2005). Longer lifespan, altered metabolism, and stress resistance in Drosophila from ablation of cells making insulin-like ligands. Proceedings of the National Academy of Sciences, 102(8), 3105-3110.
Jang, D., Kwon, H., Jeong, K., Lee, J., & Pak, Y. (2015). Essential role of flotillin-1 palmitoylation in the intracellular localization and signaling function of IGF-1 receptor. J Cell Sci, 128(11), 2179-2190.
Afschar S., Toivonen, J. M., Hoffmann, J. M. et al., 2016. Nuclear hormone receptor DHR96 mediates the resistance to xenobiotics but not the increased lifespan of insulin mutant Drosophila. Proc Natl Acad Sci USA. 113(5):1321-1326.
Partridge, L., and Gems, D. (2002). Mechanisms of ageing: public or private? Nat. Rev. Genet. 3, 165-175.
Tatar, M., Kopelman, A., Epstein, D., Tu, M. P., Yin, C. M., Garofalo, R. S., 2001. Amutant Drosophila insulin receptor homolog that extends life-span and impairs neuroendocrine function. Science 292, 107-110. Arthropod Pesticide Resistance Database. [Online]. Available: www.pesticideresistance.org [26 Jun. 2018].
Oakeshott J G, Irene Home I, Sutherland T D and Russell R J, 2003. The genomics of insecticide resistance. Genom Biol 4:202.
Barsyte, D., Lovejoy, D. A., Lithgow, G. J., 2001. Longevity and heavy metal resistance in daf-2 long-lived mutants of Caenorhabditis elegans. FASEB J, 15, 627-634.
Baumeister, R., Schaffitzel, E., Hertweck, M., 2006. Endocrine signaling in Caenorhabditis elegans controls stress response and longevity. J. Endocrinol, 190, 191-202.
Brandt A, Scharf, M., Pedra, J. H. F., et al., 2002. Differential expression and induction of two Drosophila cytochrome P450 genes near the Rst (2) DDT locus. Insect Mol Biol 11(4):337-341.
Busvine, J. R., 1957. Insecticide-resistant strains of insects of public health importance. Transactions of the Royal Society of Tropical Medicine and Hygiene, 51(1), 11-31.
Clancy, D. J., Gems, D., Harshman, L. G., Oldham, S., Stocker, H., Hafen, E., Partridge, L., 2001. Extension of life-span by loss of CHICO, a Drosophila insulin receptor substrate protein. Science, 292(5514), 104-106.
Daborn P J, Lumb, C., Boey, A., et al., 2007. Evaluating the insecticide resistance potential of eight Drosophila melanogaster cytochrome P450 genes by transgenic over-expression. Insect Biochem Mol Biol, 37(5):512-519.
Edgar, B. A., 2006. How flies get their size: genetics meets physiology. Nat. Rev. Genet. 7, 907e916.
Engelman, J. A., Luo, J., Cantley, L. C., 2006. The evolution of phosphatidylinositol 3-kinase as regulators of growth and metabolism. Nat. Rev. Genet. 7, 606e619.
Festucci-Buselli R. A., Carvalho-Dias A. S., de Oliveira-Andrade M. et al., 2005 Expression of Cyp6g1 and Cyp12d1 in DDT resistant and susceptible strains of Drosophila melanogaster. Insect Mol Biol, 14(1): 69-77.
Gellatly K. J., Yoon K. S., Doherty J. J., et al., 2015. RNAi validation of resistance genes and their interactions in the highly DDT-resistant 91-R strain of Drosophila melanogaster. Pesticide biochemistry and physiology, 121: 107-115.
Hwangbo, D. S., Gersham, B., Tu, M. P., et al., 2004. Drosophila dFOXO controls lifespan and regulates insulin signalling in brain and fat body. Nature, 429(6991), 562.
Kenyon, C., 2005. The plasticity of aging: insights from long-lived mutants. Cell 120, 449-460.
Larsen, P. L., 1993. Aging and resistance to oxidative damage in Caenorhabditis elegans. Proc. Natl. Acad. Sci. U.S.A, 90, 8905-8909.
Li, F., Hu, J., Tian, J., et al., 2016. Effects of phoxim on nutrient metabolism and insulin signaling pathway in silkworm midgut. Chemosphere, 146, 478-485.
Lithgow, G. J. White, T. M., Melov, S., et al., 1995. Thermotolerance and extended life-span conferred by single-gene mutations and induced by thermal stress. Proc. Natl. Acad. Sci. U.S.A, 92, 7540-7544
Merrell D. J., Underhill J. C., 1956. Selection for DDT resistance in inbred, laboratory and wild stocks of Drosophila melanogaster. J Econ Entomol, 49(3):300-306.
Merrell, D. J., 1960. Heterosis in DDT resistant and susceptible populations of Drosophila melanogaster. Genetics, 45(5), 573-581.
Merrell, D. J., 1965. Competition involving dominant mutants in experimental populations of Drosophila melanogaster. Genetics, 52(1), 165-189.
Murakami, S. and Johnson, T. E., 1996. A genetic pathway conferring life extension and resistance to UV stress in Caenorhabditis elegans. Genetics, 143, 1207-1218.
Pedra J, McIntyre L, Scharf M. et al., 2004. Genome-wide transcription profile of field- and laboratory-selected dichlorodiphenyltrichloroethane (DDT)-resistant Drosophila. Proc Natl Acad Sci USA. 101(18):7034-7039.
Pittendrigh B. R., Reenan R., Ffrench-Constant R. H., et al., 1997. Point mutations in the Drosophila sodium channel gene para associated with resistance to DDT and pyrethroid insecticides. Mol Gen Genet, 256(6):602-610.
Sadasivaiah S., Tozan Y., Breman J. G., 2007. Dichlorodiphenyltrichloroethane (DDT) for indoor residual spraying in Africa: how can it be used for malaria control? The American journal of tropical medicine and hygiene, 77(6_Suppl): 249-263.
Saltiel, A. R. and Kahn, C. R., 2001. Insulin signalling and the regulation of glucose and lipid metabolism. Nature, 13, 799-806.
Scott, B., Avidan, M. S., Crowder, C. M., 2002. Regulation of hypoxic death in C. elegans by the insulin/IGF receptor homolog DAF-2. Science 296, 2388-2391.
Seong, K. M., Coates, B. S., Berenbaum, M. R., et al., 2018. Comparative CYP-omic analysis between the DDT susceptible and resistant Drosophila melanogaster strains 91-C and 91-R. Pest management science 74: 2530-2543.
Yau D T, Mennear J H, 1977. The inhibitory effect of DDT on insulin secretion in mice. Toxicology and Applied Pharmacology 39: 81-88.
Kacew S, Singhal R L, 1974. Role of cyclic adenosine 39:59-monophosphate in the action of 1,1,1-trichloro-2,2-bis-(p-chlorophenyl)ethane (DDT)on hepatic and renal metabolism. Biochemical Journal 142: 145-152.
Seong, K. M., Coates, B. S., Sun, W. et al., 2017. Changes in neuronal signaling and cell stress response pathways are associated with a multigenic response of Drosophila melanogaster to DDT selection. Genome biology and evolution, 9(12), 3356-3372.
Seong, K. M., Sun, W., Clark, J. M., et al., 2016. Splice form variant and amino acid changes in MDR49 confers DDT resistance in transgenic Drosophila. Scientific reports, 6, 23355.
Everett, C. J., Frithsen, I. L., Diaz, V. A., Koopman, R. J., Simpson Jr, W. M., & Mainous III, A. G. (2007). Association of a polychlorinated dibenzo-p-dioxin, a polychlorinated biphenyl, and DDT with diabetes in the 1999-2002 National Health and Nutrition Examination Survey. Environmental research, 103(3), 413-418.
Lee Y M, Ha C M, Kim S A, et al. Persistent organic pollutants impair insulin secretory function of pancreatic beta-cells: Human and in vitro evidence. Diabetes, 2017: db170188.
Strycharz J. P., Lao, A., Li, H., et al., 2013. Resistance in the highly DDT-resistant 91-R strain of Drosophila melanogaster involves decreased penetration, increased metabolism, and direct excretion. Pestic Biochem Physiol, 107(2): 207-217.
Tatar, M. Bartke, A., Antebi, A., 2003. The endocrine regulation of aging by insulin-like signals. Science, 299, 1346-1351 Underwood, L. E., Thissen, J. P., Lemozy, S., K et al., 1994. Hormonal and nutritional regulation of IGF-I and its binding proteins. Horm. Res. 42, 145-151.
Cox S, Niskar A S, Narayan K M, Marcus M (2007) Prevalence of self-reported diabetes and exposure to organochlorine pesticides among Mexican Americans:Hispanic health and nutrition examination survey, 1982-1984.
Environ Health Perspect 115: 1747-1752.

### Example 2: Drosophila Bioassay with Cybermethrin Treated with Hydrazine Sulphate

Inhibitor Treatment and Cypermethrin Bioassay

Adult flies were placed in bottles with instant drosophila blue diet (Formula 2-24®, carolina.com) made with inhibitor solutions for two days. The solutions are, hydrazine sulphate (Hys; PEPCK inhibitor, Sigma-Aldrich, 10 mM), or lithium chloride (LiCl; GSK3β inhibitor, Sigma-Aldrich, 20 mM) (Hussain et al., 2017; Mudher et al., 2004) and both Hys plus LiCl.

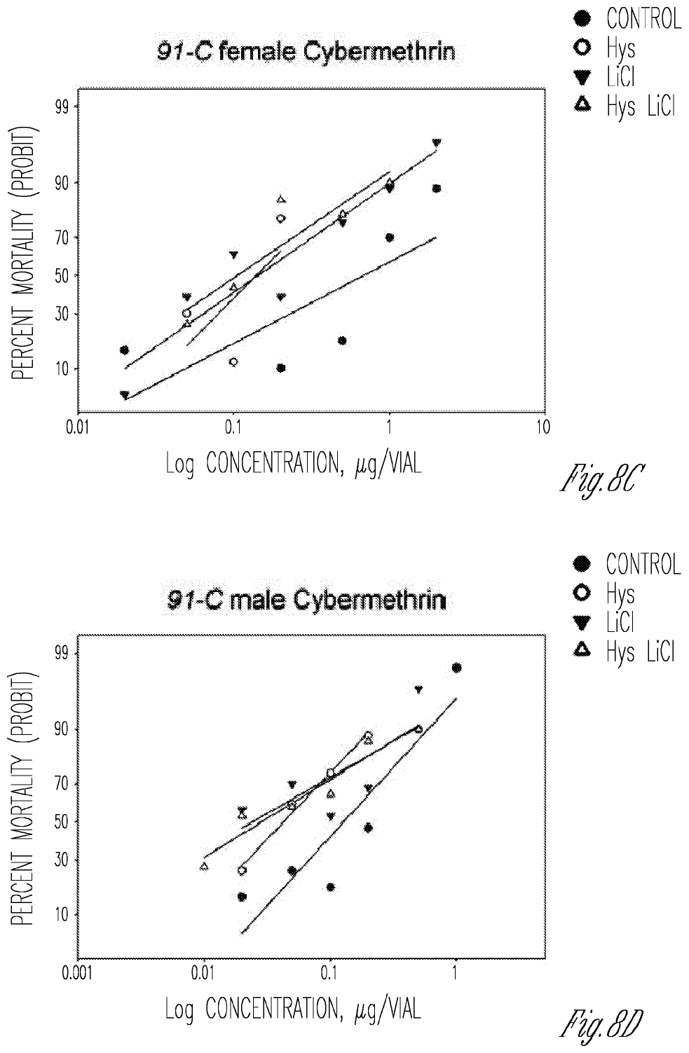

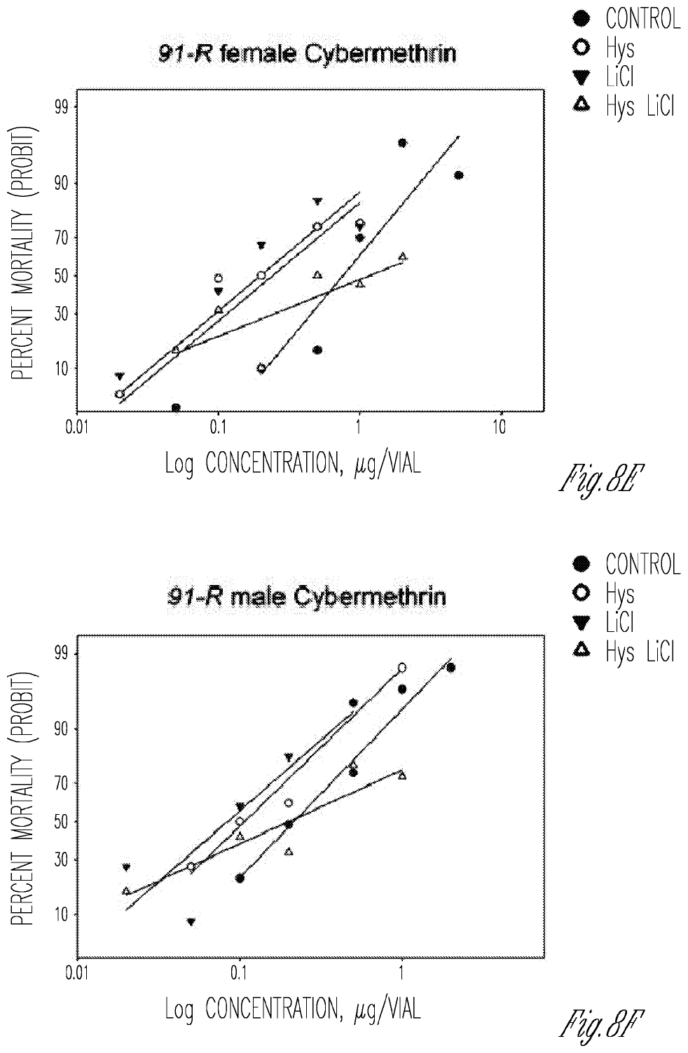

Mortality bioassays using Cypermethrin were conducted following the method of Strycharz et al. (2013). Cypermethrin were dissolved in acetone at concentrations ranging from 50, 25, 10, 5, 2.5, 1.0, 0.5, and 0.1 μg/ml from series dilution. 200 μl of each concentration of solution as well as 200 μl acetone were transferred into 20 ml transparent glass vials and rolled on its side in a fume hood till the acetone completely evaporated. 20 flies of 91C, 91R and Canton-S stains were placed into vials for either female or male. Vials were capped with cotton plugs moistened with a 5% sucrose solution in distilled water. The number of dead flies were recorded after 24 hours. For each concentration of Cypermethrin, three replicate vials were used. For each strain by sex combination, 8 concentrations of dosage were used including 0 control. A total of 8×3×20 flies were used for each of strain by sex combinations. For mortality bioassay, Probit analysis tool in SAS was used (SAS 9.4, SAS Ins, USA). See Table 5 and FIG. 8A-F

TABLE 5
LC₅₀'s of Canton-S, 91-C and 91-R for Cypermethrin
without (control) and with inhibitor feeding of Hydrazine
sulfate (Hys) or Lithium Chloride (LiCl)

| | | LD₅₀ | (95% C.I.) | | Synergism |
|---|---|---|---|---|---|
| Strain | Inhibitors | (μg/vial) | lower | upper | Ratio |
| Canton-S | Control | 0.58 | 0.51 | 0.65 | 1.00 |
| ♀ | Hys | 0.16 | 0.12 | 0.21 | 3.58 |
| | LiCl | 0.78 | 0.70 | 0.86 | 0.74 |
| | Hys+LiCl | 0.14 | 0.06 | 0.24 | 4.06 |
| Canton-S | Control | 2.1 | 1.5 | 3.4 | 1.00 |
| ♂ | Hys | 0.2 | 0.1 | 0.3 | 3.78 |
| | LiCl | 0.9 | 0.8 | 1.6 | 0.60 |
| | Hys+LiCl | 0.2 | 0.1 | 0.2 | 0.60 |
| 91-C♀ | Control | 0.66 | 0.51 | 0.87 | 1.00 |
| | Hys | 0.15 | 0.09 | 0.24 | 4.39 |
| | LiCl | 0.13 | 0.09 | 0.19 | 4.93 |
| | Hys+LiCl | 0.21 | 0.14 | 0.31 | 3.13 |
| 91-C♂ | Control | 0.15 | 0.11 | 0.20 | 1.00 |
| | Hys | 0.04 | 0.03 | 0.06 | 5.73 |
| | LiCl | 0.03 | 0.01 | 0.05 | 11.92 |
| | Hys+LiCl | 0.03 | 0.02 | 0.04 | 5.48 |
| 91-R♀ | Control | 0.79 | 0.59 | 1.04 | 1.00 |
| | Hys | 0.20 | 0.15 | 0.27 | 3.87 |
| | LiCI | 0.17 | 0.12 | 0.24 | 4.58 |
| | Hys+LiCl | 1.04 | 0.58 | 2.97 | 0.76 |
| 91-R♂ | Control | 0.23 | 0.20 | 0.27 | 1.00 |
| | Hys | 0.11 | 0.11 | 0.14 | 3.88 |
| | LiCl | 0.08 | 0.08 | 0.11 | 3.36 |
| | Tys+LiCl | 0.20 | 0.20 | 0.42 | 1.98 |

The invention is described with reference to various specific and preferred embodiments and techniques. However, it should be understood that many variations and modifications may be made while remaining within its scope. All referenced publications, patents and patent documents are intended to be incorporated by reference, as though individually incorporated by reference.

1. A method for increasing susceptibility of an insect to a pesticide comprising contacting an insect, soil, wood, plant, seeds, grain or manmade structure with one or more inhibitors of insect resistance.

2. A method of decreasing resistance of an insect to a pesticide comprising contacting an insect, soil, wood, plant, seeds, grain or manmade structure with one or more inhibitors of insect resistance.

3. A method to increase toxicity of a pesticide in an insect comprising contacting an insect, soil, wood, plant, seeds, grain or manmade structure with one or more inhibitors of insect resistance.

4. A method for providing protection against or treating a pest infestation comprising contacting a pest, soil, wood, plant, seeds, grain or manmade structure with one or more inhibitors of insect resistance.

5. A method for reducing insect resistance in a plant comprising expressing in said plant an RNA that specifically interferes with expression of an insect gene.

6. The method of claim 1, wherein the inhibitor contacts said insect, soil, wood, plant, seeds, grain or manmade structure before, after or simultaneously with one or more pesticides.

7. The method of claim 1, wherein the inhibitor modulates the activity of proteins that play a role in energy pathways or other metabolic pathways of said insect, including energy-related pathway proteins, metabolism-related pathway proteins, insulin/insulin-like growth factor (IGF)-like signaling (IIS) pathway proteins; insulin signaling pathway proteins, Phosphoenolpyruvate carboxykinase (PEPCK), Glycogen synthase kinase 3 beta (GSK3β), Lipin (Lpin-PE), Insulin-like peptide 6 (Dilp6-PD), Cchamide-2 (CCHa2-PA), Insulin-like peptide 8 (Dilp8-PB), Flotillin (Flo2-PJ), rolled (rl-PH), Phosphorylase kinase gamma subunit (PhKγ-PF), Hexokinase (Hex-C-PA), Fructose-1,6-bisphosphatase (fbp-PF), Lipin (Lpin-PL), Acetyl-coa carboxylase/biotin carboxylase 1 (ACC-PA), Glycogen synthase (GlyS-PA), and/or Glycogen phosphorylase (GlyP-PA).

8. The method of claim 1, wherein the inhibitor is one or more of hydrazine sulphate, 3-alkyl-1,8-dibenzylxanthines, oxalate and phosphonoformate, 3-mercaptopicolinic acid, (N′1-({5-[1-methyl-5-(trifluoromethyl)-1H-pyrazol-3-yl]-2-thienyl}methylidene)-2,4-dichlorobenzene-1-carbohydrazide), metformin, Beryllium, lithium chloride, dibromocantharelline, hymenialdesine, meridianin, sodium borate, and/or resorcylic acid lactone.

9. The method of claim 1, wherein the inhibitor is hydrazine sulphate and/or lithium chloride.

10. The method of claim 1, wherein the insect or pest is cotton bollworm, tobacco whitefly, two-spotted spider mite, diamondback moth, taro caterpillar, red flour beetle, green peach aphid, fall armyworm, fly, bedbugs, cockroaches, ants, termites, mites, head or body lice, rice weevils, maize weevils, and/or cotton aphid.

11. The method of claim 1, wherein the insect or pest is fall armyworm, spotted wing Drosophila, red flour beetles, and/or diamondback moths.

12. The method of claim 1, wherein the plant is a crop, flower, or forestry plant.

13. The method of claim 1, wherein the plant is a dicotyledon or monocotyledon.

14. The method of claim 5, wherein the gene codes for a protein that has a role in an energy pathway or metabolic pathway of said insect, including energy-related pathway proteins, metabolism-related pathway proteins, insulin/insulin-like growth factor (IGF)-like signaling (IIS) pathway proteins; insulin signaling pathway proteins, Phosphoenolpyruvate carboxykinase (PEPCK), Glycogen synthase kinase 3 beta (GSK3β), Lipin (Lpin-PE), Insulin-like peptide 6 (Dilp6-PD), Cchamide-2 (CCHa2-PA), Insulin-like peptide 8 (Dilp8-PB), Flotillin (Flo2-PJ), rolled (rl-PH), Phosphorylase kinase gamma subunit (PhKγ-PF), Hexokinase (Hex-C-PA), Fructose-1,6-bisphosphatase (fbp-PF), Lipin (Lpin-PL), Acetyl-coa carboxylase/biotin carboxylase 1 (ACC-PA), Glycogen synthase (GlyS-PA), and/or Glycogen phosphorylase (GlyP-PA).

15. The method of claim 5, wherein the gene is PEPCK-PA or GSK33-PO.

16. The method of claim 6, wherein the contacting is by spraying a liquid or powder on said insect, soil, wood, plant, seeds, grain or manmade structure or by ingestion by the insect and/or pest.

17. A composition comprising at least one inhibitor of insect resistance, at least one pesticide and a carrier.


SEQUENCE LISTING (36 sequences)

SEQ ID NO: 1 - DNA, 21 bp, Artificial Sequence
  A synthetic primer
ctttggattggctcgtattgc

SEQ ID NO: 2 - DNA, 21 bp, Artificial Sequence
  A synthetic primer
atcgggtagcaacgtattctg

SEQ ID NO: 3 - DNA, 21 bp, Artificial Sequence
  A synthetic primer
attcttcaaccaaatgctgcg

SEQ ID NO: 4 - DNA, 17 bp, Artificial Sequence
  A synthetic primer
ccggatggcctcctcaa

SEQ ID NO: 5 - DNA, 23 bp, Artificial Sequence
  A synthetic primer
ggctatactcaacgataccactg

SEQ ID NO: 6 - DNA, 23 bp, Artificial Sequence
  A synthetic primer
ccacattctcaacatcttcaacg

SEQ ID NO: 7 - DNA, 22 bp, Artificial Sequence
  A synthetic primer
cagtagcggtgattccaaaatg

SEQ ID NO: 8 - DNA, 19 bp, Artificial Sequence
  A synthetic primer
ggatggagttgagcagctg

SEQ ID NO: 9 - DNA, 18 bp, Artificial Sequence
  A synthetic primer
atcaacaccaagggcgag

SEQ ID NO: 10 - DNA, 19 bp, Artificial Sequence
  A synthetic primer
cgggaacagttgatcgacg

SEQ ID NO: 11 - DNA, 20 bp, Artificial Sequence
  A synthetic primer
agatccaaacccgtccaatc

SEQ ID NO: 12 - DNA, 20 bp, Artificial Sequence
  A synthetic primer
ggagttgtgattggtgttgc

SEQ ID NO: 13 - DNA, 22 bp, Artificial Sequence
  A synthetic primer
cctcacatccacaaatcctctg

SEQ ID NO: 14 - DNA, 21 bp, Artificial Sequence
  A synthetic primer
gatacccatcaccgtacactc

SEQ ID NO: 15 - DNA, 21 bp, Artificial Sequence
  A synthetic primer
ccaagacctaccagaaccaag

SEQ ID NO: 16 - DNA, 19 bp, Artificial Sequence
  A synthetic primer
gctccactccccaaatctc

SEQ ID NO: 17 - DNA, 22 bp, Artificial Sequence
  A synthetic primer
gatgacgaagccctctataacg

SEQ ID NO: 18 - DNA, 19 bp, Artificial Sequence
  A synthetic primer
attatgaggcccacatcgg

SEQ ID NO: 19 - DNA, 22 bp, Artificial Sequence
  A synthetic primer
cctcagcgatgtgatacagaag

SEQ ID NO: 20 - DNA, 21 bp, Artificial Sequence
  A synthetic primer
cagcacaaatcggttacgttc

SEQ ID NO: 21 - DNA, 19 bp, Artificial Sequence
  A synthetic primer
actatgtgcgtccgaatgg

SEQ ID NO: 22 - DNA, 22 bp, Artificial Sequence
  A synthetic primer
gggcgggatagtcattaaagtc

SEQ ID NO: 23 - DNA, 21 bp, Artificial Sequence
  A synthetic primer
gacagaaggtccatcgagttc

SEQ ID NO: 24 - DNA, 20 bp, Artificial Sequence
  A synthetic primer
ttcggctgatgtagtgatgc

SEQ ID NO: 25 - DNA, 21 bp, Artificial Sequence
  A synthetic primer
attcttcaaccaaatgctgcg

SEQ ID NO: 26 - DNA, 17 bp, Artificial Sequence
  A synthetic primer
ccggatggcctcctcaa

SEQ ID NO: 27 - DNA, 22 bp, Artificial Sequence
  A synthetic primer
cgcttcctctccattcagtatg

SEQ ID NO: 28 - DNA, 22 bp, Artificial Sequence
  A synthetic primer
gcacttttccacgtactgtttg

SEQ ID NO: 29 - DNA, 23 bp, Artificial Sequence
  A synthetic primer
gtcatcttcgtataagcaaaccg

SEQ ID NO: 30 - DNA, 21 bp, Artificial Sequence
  A synthetic primer
atcgtctgcttgatctccttg

SEQ ID NO: 31 - protein, 640 aa, D. melanogaster
MLLKGAAQLLRCGLVSPHRNSNFRLTCRFLSIQYGDAKILTPAVKQYVEKCVELCQPERVHICDGTEVESKLLQGIMLKKGTIIPLPKYENCWLARTNPADVARVEGKTFISTDSKEQTVPVTEKATPGMLGNWIAEKDMQAAIKERFPGCMKGRTMYVIPFSMGPVGSPLSKIGIEITDSPYVVESMKIMTRAGNPVLNYLQSGDGQFVKCLHSVGTPKSGVQAMPSWPCDPERTIVLHKPAENEIVSYGSGYGGNSLLGKKCLALRIGSTIAKREGWLAEHMLILGITNPQGKKIYIAAAFPSACGKTNLAMMTPTLPGYKVECVGDDIAWMKFDNKGVLRAINPENGFFGVAPGTSRATNPIAMDTIFRNSVFTNVASTSDGGVYWEGMEKDQLKGVTVTDWLGKLWSQESGKPAAHPNSRFCTPASQCPIIDPAWEDSEGVPISAILFGGRRPSGVPLVYEARDWKHGVFIGAAMRSEATAAAEFKGKVIMHDPFAMRPFFGYNFGDYLGHWLSMEQRGQVPKIFHVNWFRKSSEGKFLWPGFGENSRVLDWIFRRVEGEQCFEDSPIGRLPSKNSLNLDSLENIDLDQLFDLPKDFWEQEVAAIERRYFEEQVGHHLPGAVAEELRELKARVADM

SEQ ID NO: 32 - protein, 640 aa, D. melanogaster
MLLKGAAQLLRCGLVSPHRNSNFRLTCRFLSIQYGDAKILTPAVKQYVEKCVELCQPERVHICDGTEVESKLLQGIMLKKGTIIPLPKYENCWLARTNPADVARVEGKTFISTDSKEQTVPVTEKATPGMLGNWIAEKDMQAAIKERFPGCMKGRTMYVIPFSMGPVGSPLSKIGIEITDSPYVVESMKIMTRAGNPVLNYLQSGDGQFVKCLHSVGTPKSGVQAMPSWPCDPERTIVLHKPAENEIVSYGSGYGGNSLLGKKCFALRIGSTIAKQEGWLAEHMLILGITNPQGKKIYITAAFPSACGKTNLAMLNPTLPGYKVECVGDDIAWMKFDRKGVLRAINPENGFFGVAPGTSMETNPIAMDTIFRNSVFTNVASTSDGGVYWEGMEKDQLKGVTVTDWLGKLWTKDSGKPAAHPNSRFCTPASQCPIIDEAWEDPEGVPISAMLFGGRRPAGVPLIYEARDWTHGVFIGAAMRSEATAAAEFKGKVIMHDPFAMRPFFGYNFGDYVAHWLSMEKRGQVPKIFHVNWFRKSAEGKFMWPGYGENSRVLEWIFRRVEGEQCFEDSPIGRLPSKNSLNLDSLENIDLDQLFDLPKDFWEQEVAAIERRYFEEQVGHHLPGAVAEELRELKARVADM

SEQ ID NO: 33 - protein, 641 aa, D. melanogaster
MLLKGAAQLLRCGLVSPHRNSNFRLTCRFLSIQYGDAKILTPAVKQYVEKCVELCQPERVHICDGTEGESKLLQGIMLKKGTIIPLPKYENCWLARTNPADVARVEGKTFISTDSKEQTVPVTEKATPGMLGNWIAEKDLQAAIKERFPGCMKGRTMYVIPFSMGPVGSPLSKIGIEITDSPYVVESMKIMTRAGNPVLNYLQSGDGQFVKCLHSVGTPKSGVQAMPSWPCDPERTIVLHKPAENEIVSYGSGYGGNSLLGKKCFALRIGSTIAKQEGWLAEHMLILGITNPQGKKIYITAAFPSACGKTNLAMLNPTLPGYKVECVGDDIAWMKFDKKGVLRAINPENGFFGVAPGTSMETNPIAMDTIFRNSVFTNVASTSDGGVYWEGMEKDQLKGVTVTDWLGKLWTKDSGKPAAHPNSRFCTPASQCPIIDEAWEDPEGVPISAMLFGGRRPAGVPLIYEARDWTHGVFIGAAMRSEATAAAEFKGKVIMHDPFAMRPFFGYNFGDYVAHWLSMEKRGQVPKIFHVNWFRKSAKGKFMWPGYGENSRVLEWIFRRVEGEQCFEDSPIGRLPSKNSLNLDSLKENIDLDQLFDLPKDFWEQEVAAIERRYFEEQVGHHLPGPVAEELRELKARVADM

SEQ ID NO: 34 - protein, 772 aa, D. melanogaster
MIQETSGGQYSTLKVDKSQVVPVAVPRGVRKVVRVVRKKKLAPGSGSVNEASESDGAGSGTTTSGRQNSIDASKPPAKVIKNKRGSLGGGGAAPPIPLVTKKKNKRRSSSEEEASTGNESPIESEPESGSGSSSSSAGSESDTDSETTSSSSSTTESSNADRKSKANGKHKPLSAAAKAAQSACSSSLVVAAALKKSKPPNRSGSGASIASAGGGGKQYKDPSMKQLIGKLNDLWPEHSVALSIPKEVDRSKEKLEGAWETTGRDGSKITTVVATPGQGTDRVQEVSYTDTKVIGNGSFGVVFQAKLCDTGELVAIKKVLQDRRFKNRELQIMRKLEHCNIVKLLYFFYSSGEKLYMYQLFRSLAYIHSLGICHRDIKPQNLLLDPETAVLKLCDFGSAKQLLHGEPNVSYICSRYYRAPELIFGAINYTTKIDVWSAGCVLAELLLGQPIFPGDSGVDQLVEVIKVLGTPTREQIREMNPNYTEFKFPQIKSHPWQKVFRIRTPTEAINLVSLLLEYTPSARITPLKACAHPFFDELRMEGNHTLPNGRDMPPLFNFTEHELSIQPSLVPQLLPKHLQNASGPGGNRPSAGGAASIAASGSTSVSSTGSGASVEGSAQPQSQGTAAAAGSGSGGATAGTGGASAGGPGSGNNSSSGGASGAPSAVAAGGANAAVAGGAGGGGGAGAATAAATATGAIGATNAGGANVTGSQSNSALNSSGSGGSGNGEAAGSGSGSGSGSGGGNGGDNDAGDSGAIASGGGAAETEAAASG

SEQ ID NO: 35 - protein, 773 aa, D. melanogaster
VIQETSGGQYSTLKVDKSQVVPVAVPRGVRKVVRVVRKKKLAPGSGSVNEASESDGAGSGTTTSGRQNSIDASKPPAAKVIKNKRGSLGGGGAAPPIPLVTKKKNKPRSSSEEEASTGNESPIESEPESGSGSSSSSAGSDSDSDSETTSSSSSTTESSNADRKSKANGKHKPLSAATKAAQSACSSSLVVAAALKKSKPPNRSGSGASIASAGGGGKQYKDPSMKQLIGKLNDLWPEHSVALSIPKEVDRSKEKLEGAWETTGRDGSKITTVVATPGQGTDRVQEVSYTDTKVIGNGSFGVVFQAKLCDTGELVAIKKVLQDRRFKNRELQIMRKLEHCNIVKLLYFFYSSGEKLYMYQLFRSLAYIHSLGICHRDIKPQNLLLDPETAVLKLCDFGSAKQLLHGEPNVSYICSRYYRAPELIFGAINYTTKIDVWSAGCVLAELLLGQPIFPGDSGVDQLVEVIKVLGTPTREQIREMNPNYTEFKFPQIKSHPWQKVFRIRTPTEAINLVSLLLEYTPSARITPLKACAHPFFDELRMEGNHTLPNGRDMPPLFNFTEHELSIQPSLVPQLLPKHLQNASGPGGNRPSAGGAASIAASGSTSVSSTGSGASVEGSAQPQSQGTAAAAGSGSGGATAGTGGASAGGPGSGNNSSSGGASGAPSAVAAGGANAAVAGGAGGGGGAGAATAAATATGAIGATNAGGANVTGSQSNSALNSSGSGGSGNGEAAGSGSGSGSGSGGGNGGDNDAGDSGAIASGGGAAETEAAASG

SEQ ID NO: 36 - protein, 772 aa, D. melanogaster
VIQETSGGQYSTLKVDKSQVVPVAVPRGVRKVVRVVRKKKLAPGSGSVNEASESDGAGSGTTTSGRQNSIDASKPPAKVIKNKRGSLGGGGAAPPIPLVTKKKNKRRSSSEEEASTGNESPIESEPESGSGSSSSSAGSESDTDSETTSSSSSTTESSNADRKSKANGKHKPLSAAAKAAQSACSSSLVVAAALKKSKPPNRSGSGASIASAGGGGKQYKDPSMKQLIGKLNDLWPEHSVALSIPKEVDRSKEKLEGAWETTGRDGSKITTVVATPGQGTDRVQEVSYTDTKVIGNGSFGVVFQAKLCDTGELVAIKKVLQDRRFKNRELQIMRKLEHCNIVKLLYFFYSSGEKLYMYQLFRSLAYIHSLGICHRDIKPQNLLLDPETAVLKLCDFGSAKQLLHGEPNVSYICSRYYRAPELIFGAINYTTKIDVWSAGCVLAELLLGQPIFPGDSGVDQLVEVIKVLGTPTREQIREMNPNYTEFKFPQIKSHPWQKVFRIRTPTEAINLVSLLLEYTPSARITPLKACAHPFFDELRMEGNHTLPNGRDMPPLFNFTEHELSIQPSLVPQLLPKHLQNASGPGGNRPSAGGAASIAASGSASVSSTGSGASVEGSAQPQSQGTAAAAGSGSGGATAGTGGASAGGPGSGNNSSSGGASGAPSAVAAGGANAAVAGGAGGGGGAGAATAAATATGAIGATNAGGANVTGSQSNSALNSSGSGGSGNGEAAGSGSGSGSGSGGGNGGDNDAGDSGAIASGGGAAETEAAASG